(19) United States
(12) Patent Application Publication
(10) Pub. No.: US 2025/0369883 A1
(43) Pub. Date: Dec. 4, 2025

(54) METHODS AND DEVICES FOR FLUORESCENCE-BASED ANALYTE DETECTION

(71) Applicant: Siomyx, Inc., Redwood City, CA (US)

(72) Inventors: Arjang HASSIBI, Palo Alto, CA (US); Mark William MCDERMOTT, Austin, TX (US); Edmond KU, Sunnyvale, CA (US)

(21) Appl. No.: 19/203,715

(22) Filed: May 9, 2025

Related U.S. Application Data
(63) Continuation of application No. PCT/US2023/037094, filed on Nov. 9, 2023.
(60) Provisional application No. 63/424,295, filed on Nov. 10, 2022.

(51) Int. Cl. G01N 21/64

(52) U.S. Cl. CPC G01N 21/6408; G01N 21/6486; G01N 2021/6471

(57) ABSTRACT

The present disclosure provides methods, devices, and systems for fluorescence-based analyte detection. Devices may include a surface layer configured to be in contact with a solution. The surface layer may include an immobilized capture probe configured to bind an analyte. The device may include a photodiode transducer, current switch, or circuitry. The photodiode transducer may include a first photodiode disposed adjacent to a second photodiode. The current switch may divert current to a high gain detection path or a low gain detection path. Methods may include using the devices and systems described herein for analyte detection.

### CROSS-REFERENCE

This application is a continuation of International Application No. PCT/US2023/037094, filed Nov. 9, 2023, which claims the benefit of U.S. Provisional Application No. 63/424,295, filed Nov. 10, 2022, each of which is incorporated by reference herein in its entirety.

### BACKGROUND

Array based integrated sensors may combine semiconductor-based sensor arrays and addressable molecular capturing arrays (e.g., microarrays). See References 1-2. Array based sensors may use complex transduction methods, such as continuous-wave fluorescent-based spectroscopy. Integration of molecular constructs (e.g., fluorescence tags and acceptor-donor moieties) into these types of systems may aim to generate analyte specific fluorescence signals. See References 3-7. In such systems, different sensor arrangements may offer different sets of performances in terms of analytical sensitivity, time-to-results, cost of manufacturing, form factor, array density, and size.

### SUMMARY

Recognized herein is a need for improved systems, devices, and methods for identification and quantification of analytes. Such systems may include detection methods integrated with semiconductor-based optical sensor devices to identify and quantify analytes, for example, in an aqueous sample. Methods and devices described herein may provide low-cost, high-performance array-based sensors by using differential time-resolved photonic transducers that may be coupled to differential photosensors that may permit analyte detection via time-resolved fluorescence detection. The sensors described herein may be manufactured via simple and low-cost processes analogous to those used in semiconductor manufacturing. The sensors described herein may use differential photo-sensing to desensitize analyte detection from manufacturing variabilities and improve analytical sensitively and dynamic range.

An aspect of the present disclosure provides a device for time-gated detection of a presence or absence of an analyte in a solution, comprising: a biochip comprising: a surface layer comprising at least one immobilized capture probe specific for the analyte; a first optical transducer in optical communication with the surface layer; a second optical transducer disposed adjacent to the first optical transducer; an optical cover disposed over the second optical transducer; and circuitry configured to: collect, by the first optical transducer, a first optical signal from the surface layer generated upon exposure of the surface layer to a light source, and convert the first optical signal to a first electrical signal, collect, by the second optical transducer, a second optical signal, and convert the second optical signal to a second electrical signal, and generate an output signal derived at least in part from a differential of the first and second electrical signals, wherein the output signal is associated with the presence or absence of the analyte.

In some embodiments, the light source is configured to synchronize with the biochip and emit a pulse of excitation energy, wherein the pulse of excitation energy comprises a first duration of time (tₚ), a duty of cycle of the plurality of the pulses of excitation energy is no more than 50%; the first optical signal comprises a fluorescence signal having a relaxation lifetime (τ_{F}); and the first duration of time (tₚ) is about 0.1% to about 50% of the relaxation lifetime (τ_{F}). In some embodiments, the light source is configured to synchronize with the biochip and emit a plurality of pulses of excitation energy, wherein: each pulse of excitation energy of the plurality of the pulses of excitation energy comprises a first duration of time (tₚ), a duty of cycle of the plurality of the pulses of excitation energy is no more than 50%; the first optical signal comprises a fluorescence signal having a relaxation lifetime (τ_{F}); and the first duration of time (tₚ) is about 0.1% to about 50% of the relaxation lifetime (τ_{F}). In some embodiments, the biochip further comprising a current switch operably connected to the first optical transducer and the second optical transducer, wherein the current switch is configured to: divert the first and second electrical signals to a low gain detection path during a first time period when the light source is on; and divert the first and second electrical signals to a high gain detection path during a second time period when the light source is off. In some embodiments, the first optical transducer and the second optical transducer are separated by a distance about 100 nanometers (nm) to about 1 millimeter (mm). In some embodiments, the first optical transducer and the second optical transducer are substantially identical. In some embodiments, the first optical transducer is a first photodiode, a first photogate, or a first photo-resistive device. In some embodiments, the second optical transducer is a second photodiode, a second photogate, or a second photo-resistive device. In some embodiments, the first optical transducer is a first photodiode, and wherein the second optical transducer is a second photodiode. In some embodiments, the device further comprises an optical cover disposed over the second optical transducer, wherein the optical cover is configured to reduce an amount of photons emitted by the light source from contacting the second optical transducer as compared to an optical transducer without the optical cover. In some embodiments, the optical cover comprises a metal. In some embodiments, the metal is aluminum, copper, gold, lead, platinum, silver, tin, titanium, tungsten, or another metal or a metal alloy that is used in the manufacturing of semiconductor devices. In some embodiments, the metal alloy is titanium-tungsten or alloy 42. In some embodiments, the device does not include an emission filter. In some embodiments, the surface layer comprises a linker molecule configured to immobilize the capture probe. In some embodiments, the device further comprises one or more optical isolators disposed adjacent to the first and/or second optical transducers, and the one or more optical isolators are configured to direct photons to the photodiode transducer. In some embodiments, the one or more optical isolators comprise another metal. In some embodiments, the device is one of a plurality of devices, and the one or more isolators are configured to optically isolate the device from another device of the plurality of devices. In some embodiments, the device does not include an emission filter and/or an optical filter. In some embodiments, the biochip further comprising: a differential sensor circuitry configured to detect and quantize the first and second optical signals.

Another aspect of the present disclosure provides a method for time-gated detection of a presence or absence of an analyte in a solution, comprising: (a) directing the solution to a device comprising: a biochip synchronized with a light source operably coupled to the biochip, the biochip comprising: a surface layer comprising at least one immobilized capture probe specific for the analyte, a first optical transducer in optical communication with the surface layer, a second optical transducer disposed adjacent to the first optical transducer, and an optical cover disposed over the second optical transducer; (b) collecting, by the first optical transducer, a first optical signal from the surface layer generated upon exposure of the surface layer to the light source, and converting the first optical signal to a first electrical signal; (c) collecting, by the second optical transducer, a second optical signal, and converting the second optical signal to a second electrical signal; and (d) generating an output signal derived at least in part from a differential of the first and second electrical signals, wherein the output signal is associated with the presence or absence of the analyte.

In some embodiments, the method further comprises: modulating the light source and emitting a plurality of pulses of excitation energy, wherein: each pulse of excitation energy of the plurality of the pulses of excitation energy comprises a first duration of time (tₚ); a duty of cycle of the plurality of the pulses of excitation energy is no more than 50%; the first optical signal comprises a fluorescence signal having a relaxation lifetime (T_{F}); and the first duration of time (tₚ) is about 0.1% to about 50% of the relaxation lifetime (T_{F}). In some embodiments, the method comprises: diverting, via a current switch operably connected to the first and second optical transducers, the first and second electrical signals to a low gain detection path during a first time period when the light source is on; and diverting, via the current switch, the first and second electrical signals to a high gain detection path during a second time period when the light source is off. In some embodiments, the method comprises: providing a low gain digital output (Y_{L}) of a first output electrical signal based in part of the low gain detection path, and providing a high gain digital output (Y_{H}) of a second output electrical signal based in part of the high gain detection path. In some embodiments, the method further comprises: providing a calibrated digital output (Y_{C}) as the output signal, wherein

$${Y}_{C} = \frac{{Y}_{H}}{{Y}_{L}} .$$

In some embodiments, the calibrated digital output (Y_{C}) is substantially not a function of excitation photon flux (F_{X}) of an excitation light emitted by the light source. In some embodiments, the method comprise: repeating (b)-(d) one or more times. In some embodiments, the plurality of pulses of excitation energy is pulsed at least 10 times for each repeat of (b)-(d). In some embodiments, the first optical signal is generated by a fluorescent reporter molecule associated with the analyte or the immobilized capture probe. In some embodiments, the fluorescent reporter molecule has a fluorescence lifetime of greater than or equal to 100 nanoseconds (ns). In some embodiments, the fluorescence lifetime is greater than or equal to 1 microseconds. In some embodiments, the optical signal in (d) is substantially not correlated to a dark current of the first optical transducer. In some embodiments, the method further provides detecting and quantizing the first and second optical signals in (d) using a differential sensor circuitry of the biochip. In some embodiments, the method does not comprise correlated double sampling. In some embodiments, the method uses the device disclosed herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a block diagram of an example sensor system using a fluorescent-based transduction method;

FIG. 2A-2C show example differential photodiode transducers and circuit schematics;

FIG. 2A shows an example differential photodiode and circuit schematic; FIG. 2B shows an example array of eight photodiode transducers and circuit schematic; and FIG. 2C shows an example array of three photodiode transducers and circuit schematic;

FIG. 3 shows an example excitation pulse components for time-resolved fluorescence detection;

FIG. 4 shows an example excitation pulse in a pulse-modulated mode for time-resolved fluorescence detection;

FIG. 5 shows an example current switch and timing diagram;

FIG. 6 shows an example current switch with stabilization switches;

FIG. 7 shows an example sensing array and pixel cross-section;

FIG. 8 shows an example block diagram and timing diagram of an example detection circuit;

FIG. 9 shows another example block diagram and timing diagram of another example detection circuit;

FIG. 10 shows another example block diagram and timing diagram of another example detection circuit; and

FIG. 11 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “fluorescence-based detection” as used herein generally refers to a detection scheme that uses a wavelength-specific optical excitation light source to excite fluorophore constructs that may subsequently re-emit light in a different wavelength. A fluorescence detection device or instrument (e.g., fluorescence sensor) may measure the emission signal, which may represent the quantity of the fluorophore construct, in the presence of a much larger excitation signal.

The term “analyte,” as used herein, generally refers to a molecular species to be detected. Non-limiting examples include small molecules, such as organic compounds drugs, hormones, lipids, steroids, or metabolites; polynucleotides such as deoxyribonucleic acid (DNA) molecules, ribonucleic acid (RNA) molecules, and peptide nucleic acid (PNA) molecules; polypeptides such as proteins, peptides, antibodies, antigens, enzymes, and receptors; as well as tissues, organelles, and other receptor probes.

The term “probe” or “capture probe” may be used interchangeably and generally refers to a molecular species or other markers that can bind and/or interact to a specific analyte. Probes can comprise molecules and can be bound to the substrate, molecules, or other solid surface, directly or via a linker. Non-limiting examples of linkers include amino acids, polypeptides, nucleotides, oligonucleotides, and chemical linkers. A plurality of probes can be immobilized to a substrate, molecule or other solid surface and can be referred to as a probe array. A plurality of probes of a probe array may be arranged uniformly, for example as an arrangement of spots, or non-uniformity.

The term “reporter” or “reporter molecule” as used herein, generally refers to a molecular structure that can be attached to a molecule (e.g., an analyte or a probe), to permit detection of molecule, distinguishable, or traceable by providing a characteristic which may not be intrinsic to the analyte molecule. Examples of labels may include luminescent molecules (e.g., fluorophores), reduction-oxidation (redox) species, or enzymes. In some cases, labels may comprise fluorophores with long lifetimes, such as, for example, lanthanide chelates and transition metal chelates, which may be luminescent or phosphorescent.

The term “nucleotide,” as used herein, generally refers to a molecule that can serve as the monomer, or subunit, of a nucleic acid, such as deoxyribonucleic acid (DNA) or ribonucleic acid (RNA). A nucleotide may be a deoxynucleotide triphosphate (dNTP) or an analog thereof (e.g., a molecule having a plurality of phosphates in a phosphate chain, such as 2, 3, 4, 5, 6, 7, 8, 10, or more phosphates). A nucleotide may generally include adenosine (A), cytosine (C), guanine (G), thymine (T) and uracil (U), or variants thereof. A nucleotide may include any subunit that can be incorporated into a growing nucleic acid strand. Such subunit can be an A, C, G, T, or U, or any other subunit that is specific to one or more complementary A, C, G, T, or U, or complementary to a purine (e.g., A or G, or variant thereof) or a pyrimidine (e.g., C, T, or U, or variant thereof). A subunit can enable individual nucleic acid bases of group of bases (e.g., AA, TA, AT, GC, CG, CT, TC, GT, TC, AC, CA, or uracil counterparts thereof) to be resolved. A nucleotide may be labeled or unlabeled. A labeled nucleotide may yield a detectable signal, such as an optical, electrostatic, or electrochemical signal.

The terms “polynucleotide,” “oligonucleotide,” “nucleotide,” “nucleic acid,” and “nucleic acid molecule” generally refer to a polymeric form of nucleotides (polynucleotides) of various lengths, either ribonucleotide (RNA) or deoxyribonucleotides (DNA). Examples of nucleotide sequences are sequences corresponding to natural or synthetic RNA or DNA including genomic DNA and messenger RNA. The length of the sequence can be any length that can be amplified into nucleic acid amplification products, or amplicons, for example, up to about 20, 40, 100, 200, 300, 400, 500, 600, 00, 800, 21000, 1200, 1500, 2000, 5000, 12000, or more than 10000 nucleotides in length.

The terms “peptide,” “polypeptide,” and “protein” as used herein generally refer to a compound comprising amino acid residues covalently linked by peptide bonds. Polypeptides may include any peptide or protein comprising two or more amino acids joined to each other by peptide bonds. Examples of polypeptides may include, for example, biologically active fragments, substantially homologous polypeptides, oligopeptides, homodimers, heterodimers, polypeptides and variants thereof, modified polypeptides, derivatives, analogs, fusion proteins, or combinations thereof. A polypeptide may be a natural peptide, a recombinant peptide, or a combination thereof.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Fluorescence-Based Analyte Detection

In an aspect, the present disclosure provides system and devices for analyte detection. A device for analyte detection may be used for detecting an analyte in a solution. The device may include a surface layer, a first optical transducer, a second optical transducer, circuitry, or a combination thereof. The surface layer may be configured to be in contact or a surface layer in contact with a solution. The surface layer may include an immobilized capture probe configured to bind or bound to the analyte.

As used herein, the term “optical transducer” generally refers to a device based on optical transduction of a signal consisting of ultraviolet (10-390 nm), visible (390-700 nm), and infrared (700 nm-1 mm) spectrophotometry in transmission. The optical transducer may convert a light ray or an optical signal into an electrical signal. The optical transducer can be called as a photoelectric transducer. The optical transducer can be classified as photo emissive, photoconductive and photovoltaic transducers. The photo emissive devices may operate on the principle that radiation falling on a cathode causes electrons to be emitted from the cathode surface. The photoconductive devices may operate on the principle that whenever a material is illuminated, its resistance changes. The photovoltaic cells may generate an output voltage that is proportional to the radiation intensity. The radiation that is incident may be x-rays, gamma rays, ultraviolet, infrared or visible light. The purpose of an optical transducer is to measure a physical quantity of light and, depending on the type of transducer, then translates it into a form that is readable by an integrated measuring device.

The optical transducer can be a photodiode transducer. The photodiode transducer may be in optical communication with the surface layer. The photodiode transducer may include a first photodiode disposed adjacent to a second photodiode where the second may not be in in optical communication with the surface layer. The circuitry may be configured to or may (i) collect an optical signal from the surface layer generated upon exposure of the surface layer to an excitation light source and (ii) convert the optical signal to a first electrical output signal and a second electrical output signal using the first photodiode and the second photodiode, respectively. The first and second electrical output signals may be usable to or may be used to determine a presence or absence of the analyte.

In another aspect, a device for analyte detection may include a surface layer, photodiode transducer, current switch, circuitry, or any combination thereof. The surface layer may be configured to be in contact or may be in contact with a solution. The surface layer may comprise an immobilized capture probe configured to bind or bound to an analyte. The photodiode transducer may be in optical communication with the surface layer. The current switch may be in electrical communication with the photodiode transducer. The current switch may be configured to divert or may divert current to a high gain detection path or a low gain detection path. The circuitry may be configured to or may (i) collect an optical signal from the surface layer generated upon exposure of the surface layer to an excitation light source, (ii) convert the electrical signal to an electrical signal using said photodiode transducer, and (iii) selectively divert the electrical signal to the high gain detection path in absence of a light from the excitation light source to generate a high gain output signal and to a low gain detection path in presence of the light to generate a low gain output signal. The high gain output signal and low gain output signal may be usable to determine a presence or absence of the analyte.

In another aspect, the present disclosure provides a device for detecting an analyte. The device may include a surface layer comprising a capture probe and a photodiode transducer comprising a first photodiode disposed adjacent to a second photodiode. The second photodiode may be substantially not in optical communication with the surface. The capture probe may be configured to bind or may bind the analyte. The first photodiode and the second photodiode may be configured to convert or may convert and optical signal from the surface layer to a first electrical output signal and a second electrical output signal, respectively. The first and second electrical output signals may be usable to determine a presence or absence of the analyte.

In another aspect, the present disclosure provides a device for detecting an analyte. The device may include a surface layer comprising an immobilized capture probe, a photodiode transducer, and a current switch. The capture probe may be configured to bind or may bind the analyte. The photodiode transducer may be configured to convert or may convert an optical signal from said surface layer to an electrical signal. The current switch may be configured to divert or may divert the electrical signal to a high gain detection path or a low gain detection path to generate a high gain output signal and a low gain output signal, respectively. The high gain and low gain output signals may be usable to determine a presence or absence of the analyte.

In another aspect, the present disclosure provides methods for detecting analyte in a solution. The method may include directing a solution to a device. The device may include a surface layer and a photodiode transducer. The surface layer may comprise an immobilized capture probe configured to bind the analyte. The photodiode transducer may be in optical communication with the surface layer and may include a first photodiode and a second photodiode. The method may include directing a light from a light source to the surface layer to generate an optical signal. The optical signal may be converted to a first electrical output signal and a second electrical output signal using the first photodiode and the second photodiode, respectively. The method may further include using the first electrical output signal and the second electrical output signal to determine a presence or absence of the analyte in the solution. Directing the light from the light source to the surface layer, converting the optical signal to an electrical signal, and using the electrical output signals may be repeated one or more times to determine a presence or absence of one or more analytes.

In another aspect, the present disclosure provides methods for detecting an analyte in a solution. The method may include directing a solution to a device. The device may include a surface layer, a photodiode transducer, and a current switch. The surface layer may include an immobilized capture probe configured to bind the analyte. The photodiode transducer may be in optical communication with the surface layer. The current switch may be in electrical communication with the photodiode transducer. The current switch may divert current to a high gain detection path or a low gain detection path. The method may include directing a light from a light source to the surface layer to generate an optical signal. The optical signal may be converted to an electrical signal using the photodiode transducer. The current switch may be used to selectively diver the electrical signal to the high gain detection path in absence of the light to generate a high gain output signal and to the low gain detection path in presence of the light to generate a low gain output signal. The high gain output signal and low gain output signal may be use dot determine a presence or absence of the analyte in the solution. Directing the light from the light source to the surface layer, converting the optical signal to an electrical signal, and using the electrical output signals may be repeated one or more times to determine a presence or absence of one or more analytes.

For sensing applications, the fluorophore constructs may be incorporated into a probe-analyte moiety such that capturing of or interaction between the probe and the analyte may result in a detectable fluorescence emission signal that may be distinguishable from the excitation signal. Sensing arrays may include different probe structures (e.g., nucleic acid sequences, aptamers, antibodies, etc.) at different coordinates of an addressable planar array (e.g., a pixel) to interrogate a sample for the presence, absence, or quantity of different analytes. Measurements may be carried out by applying an excitation light source across the array and measuring the fluorescence signal for each pixel individually. Semiconductor-integrated sensing arrays may include the a probe array disposed on a top surface of a passivated semiconductor chip. The probe may be immobilized using a linker molecule and various attachment chemistries. The semiconductor chip may include an embedded fluorescence sensor array with a plurality of detection pixels. Systems for analyzing aqueous samples may further combine or integrate semiconductor chips with interface fluidic structure and devices. For example, fluidic systems may include reaction chambers, incubation chambers, fluidic inlets and outlets, bubble traps, fluidic pumps, valves, or any combination thereof. The fluidic structures may be independent of the sensing application and may be designed and implemented to not interfere with the fluorescence detection method and sensor electronics.

Fluorescence-based analyte detection may include various detection methods integrated with semiconductor-based optical sensor devices. Sensors may be configured for continuous wavelength detection or time-resolved fluorescence (TRF) detection. Alternatively, or in addition to, sensors may be configured for time-resolved fluorescence detection and may include differential time-resolved photonic transducers coupled to differential photosensors. In an example, sensors may be planar and addressable. Sensors may be placed on silicon-based integrated circuits that may be manufactured using complementary metal-oxide-semiconductor (CMOS) processes. Manufacturing processes for TRF detection may use simpler processes when comparted with continuous-wave fluorescence-based systems.

The output signal of a fluorescence-based sensor may be a measurable electrical signal (e.g., electrical current or voltage) that may be produced by an optical transducer (e.g., photodiode, photogate or photo-resistive device). In an example, the output signal of an example fluorescence-based sensor may be a measured current (e.g., I_{D}) from a photodiode. In an example photodiode, measured current may include two components. One component may be photon-induced current (e.g., photocurrent, Iₚₕ). Another component may be the dark current (e.g., I_{dc}), which may not be a function of the excitation light. See, for example, Equation (1).

$${I}_{D} = {I}_{ph} + {I}_{dc} \qquad ( 1 )$$

Equation (1) may be rewritten as a function of the excitation photon flux, Fₓ, as shown in Equation (2).

$${I}_{D} = {F}_{X} ( {θ}_{X} + n {θ}_{Y} {θ}_{E} ) + {I}_{dc} \qquad ( 2 )$$

where θ_{X} and θ_{E} may be conversion gains of excitation and emission photons, respectively, that have different wavelengths to I_{D}, n may be the concentration or surface density of the fluorophores, and θ_{Y} may be the external quantum yield of the fluorophore.

The external quantum yield of the fluorophore may be low for most fluorophore constructs, for example, excitation photons may not result in emission of photons efficiently. Therefore, to measure a small level of n using the measured current, the excitation light component (e.g., F_{X}θ_{X}) may be suppressed. In some examples, an emission filter disposed between the photodiode and the fluorophores to block the wavelengths of the excitation photon flux may suppress the excitation light component while permitting the emission wavelengths to pass through. An emission filter specific for the excitation wavelength may generate an excitement conversion gain which is significantly smaller than an emission conversion gain (e.g., θ_{X}<<θ_{E}). Using an emission filter, the excitation term of Equation (2) may be reduced to zero, as shown in Equation (3):

$${I}_{D} \approx n {F}_{X} {θ}_{Y} {θ}_{E} + {I}_{dc} \qquad ( 3 )$$

In Equation (3), the dark current is not proportional to n and may be considered a non-informative background value to be removed. The dark current term may be removed from the output using various methods, for example, correlated double sampling (CDS). See References 1, 3 and 8. Such techniques may be relied on as the dark current is independent of the excitation photon flux and may involve, for example, taking measurements in absence of the excitation light source and subtracting the measured value (e.g., I_{D0}) from Equation (3) to produce Equation (4), which is independent of the dark current.

$$Δ {I}_{D} = {I}_{D} - {I}_{D 0} \approx n {F}_{X} {θ}_{T} {θ}_{E} \qquad ( 4 )$$

Methods, such as CDS, may be difficult and costly to implement in array-based sensing application in semiconductor chips. For example, emission filters may be difficult to implement. To suppress the excitation light and arrive at Equation (3), a ration of emission conversion gains to excitation conversion gains may be greater than 10⁶ (e.g., θ_{E}/θ_{X}≥10⁶). This may be achieved using optical interference filters. See Reference 9. Optical interference filters may be sensitive to angle-of-incident (AOI) may function better when an excitation light source is collimated. In array-based sensing applications, light may pass through an aqueous environment. The aqueous environment may scatter the excitation source and a partially collimated excitation photon flux, Fₓ. In aqueous environments, emission filtering may become more difficult as blocking excitation light, scattered light, and stray light rays may improve sensing. For example, emission filters may be overdesigned to achieve θ_{E}/θ_{X}≥10⁸ (see Reference 1 and 3) or designed to be angle-insensitive using, for example, metallic light absorbing material (see Reference 10) or light absorbing coatings (e.g., organic coatings) (see Reference 11). As such, sensors including emission filters may be more complex than similar systems without emission filters.

Manufacturing processes for sensor arrays with emission filters may be more complex and incompatible with semiconductor-type manufacturing processes. The materials and processes that are used to generate emission filters (see Reference 9) may not be more complex than semiconductor-type manufacturing processes. For example, integration of emission filters into CMOS devices, which may be used for computing, communication and consumer electronics application, may use non-standard processes that increase the manufacturing costs of such devices.

It may be challenging to generate and maintain perfect uniformity of excitation photon flux across a two-dimensional (2D) array. For example, the excitation photon flux may lack uniformity and may fluctuate temporally. Excitation photon flux may have a systematic gradient across the array or a probabilistic variation at each sensor (e.g., pixel). Additionally, bubbles, debris, or any floating particles in an aqueous sample may temporarily obscure, permanently block, or scatter excitation photon flux for one or more sensors (e.g., pixels). This type of interference may increase the difficulty of estimating the concentration (e.g., n) of fluorophores using Equation (4), particularly as the excitation photon flux may also vary. Various methods may increase uniformity of excitation photon flux across the array. For example, redundant sensors (e.g., pixels) may be placed at different coordinates across the array. See References 1 and 3. However, such methods for estimating the concentration of fluorophores may be complicated as the estimates may be based on indirect measurements. Additionally, techniques like CDS may slow down measurement time. For example, CDS methods may be used to remove the dark current term from Equation (3) at the price of doubling the sample time due to the use of two identical measurements for one dark current free measurement. This technique effectively reduces the measurement speed by half, while assuming that the system remains identical between the two measurements.

The systems, devices, and methods of the present disclosure may be used to detect, analyze, or quantify a plurality of analytes present in a aqueous sample through time-resolved transduction methods. Devices may include complementary metal-oxide-semiconductor (CMOS) chips integrated into a sensor array with addressable locations. Each addressable location may comprise an independently operating photo-sensor that detects fluorescence signals from a dedicated sensing area. The sensing may be conducted in real-time and in the presence of an aqueous sample, or when such a sample is washed away after binding of an analyte to a capture probe.

The analyte sensing system may include, but is not limited to, a sensor array, reaction chamber, excitation source, controllable fluidic system, temperature controller, heaters, reagents and reporter constructs, and a digital or computer system. The sensor array may be 2D array configured to detect analytes by interfacing a top surface (e.g., surface layer) with a solution containing or suspected of containing an analyte.

The reaction chamber may provide the interface between the sample fluid (e.g., a fluidic aqueous sample that includes the analytes) with the sensor array. The reaction chamber may have any volume usable for detection of an analyte. For example, the reaction chamber may have a volume from about 0.1 microliters (pμ) to 10,000 μL. In another example, the reaction chamber may have a volume from about 1 μL to about 100 μL. The reaction chamber may include a plurality of inlets and outlets to permit interfacing with a controllable fluidic system.

The excitation source may introduce wavelength specific photon flux into the reaction chamber and toward the surface of the sensor array in a controlled and synchronized operation. The excitation source may comprise an optical light source that can create a wavelength selective photon flux with a controllable and time-varying amplitude. The light source may illuminate the sensing layer of the device and the coordinates in which signal transduction may take place. The excitation source center wavelength may be from about 200 nanometers (nm) to 1500 nm. In an example, the excitation source center wavelength may be from about 300 nm to 800 nm. The excitation source special span (e.g., bandwidth) may be from about 1 nm to 500 nm. In an example the bandwidth may be from about 10 nm to 100 nm. The excitation source photon flux may be directional and may be optically collimated. Alternatively, the excitation source may not be optically collimated. The excitation source peak output power may be from about 10 milliwatts (mW) to 100 watts (W). In an example, the excitation source peak output power may be from about 100 mW to 10 W. The excitation source may be capable of pulsing at a frequency of about 10 GHz (e.g., turning on and off in 0.1 nanoseconds (ns) to about 100 MHz (e.g., turning on and off in 0.01 microsecond (μs)), or at a frequency lower than 100 MHz. The excitation source may be capable of pulsing at a frequency of about 10 GHz, 9 GHz, 8 GHz, 7 GHz, 6 GHz, 5 GHz, 4 GHz, 3 GHz, 2 GHz, 1 GHz, 900 MHz, 800 MHz, 700 MHz, 600 MHz, 500 MHz, 400 MHz, 300 MHz, 200 MHz, 100 MHz. The excitation source may be capable of pulsing at a frequency of about 9 GHz to about 10 GHz, about 8 GHz to about 9 GHz, about 7 GHz to about 8 GHz, about 6 GHz to about 7 GHz, about 5 GHz to about 6 GHz, about 4 GHz to about 5 GHz, about 3 GHz to about 4 GHz, about 2 GHz to about 3 GHz, about 1 GHz to about 2 GHz, about 900 MHz to about 1 GHz, about 800 MHz to about 900 MHz, about 700 MHz to about 800 MHz, about 600 MHz to about 700 MHz, about 500 MHz to about 600 MHz, about 400 MHz to about 500 MHz, about 300 MHz to about 400 MHz, about 200 MHz to about 300 MHz, about 100 MHz to about 200 MHz, about 90 MHz to about 100 MHz, about 80 MHz to about 90 MHz.

The controllable fluidic system may be configured to direct fluid to or remove fluid from or may direct fluid to or remove fluid from the sensor array, including the sample or reagents, in a controlled and synchronized operation. Methods described herein may include using the controllable fluidic system to direct fluid to or from the reaction chamber. The controllable fluidic system may be used to execute the workflow and/or processes for detection and analysis of an analyte. The workflow and sequence of each fluidic operation may be selected based on the assaying method and may be, for example, flow-through and mono-directional or closed-tube. The controllable fluidic system may use fluidic components such as pumps, valves, and tubing to perform the workflow.

The temperature controller may be configured to set the temperature or may set the temperature of the reaction chamber. Methods may include using the temperature controller to set and maintain a specific temperature of the fluid of the reaction chamber or generate a temperature profile for heating or cooling. A temperature controller may include a feedback control system that measures the temperature, using temperature sensor with tin the sensor array or sensor devices coupled with the reaction chamber (e.g., a thermistor or thermocouple) and, based on the measured temperature, add or remove heat from the reaction chamber using heaters or thermal devices (e.g., Peltier devices or resistive heaters). The system may include a single heater or a plurality of heaters. The heater(s) may be integrated into the system or into the sensing array. In an example, the heater(s) are resistive-type heater(s). Temperature controllers may comprise heat sings for removing heat. Temperature controllers may have components within the sensor array or external to the sensor array. Temperature controllers may change the temperature of a substrate, reaction chamber, or sensor array. The rate of temperature change may be about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20° C. per second. The rate of temperature change can be at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20° C. per second. The rate of temperature change can be at most about 0.1, 0.2, 0.3, 0.4, 0.5, 0.6, 0.7, 0.8, 0.9, 1, 2, 3, 4, 5, 6, 7, 8, 10, 11, 12, 13, 14, 15, 16, 17, 18, 19, or 20° C. per second Temperature controllers can change temperature at a linear rate (e.g., 5° C. per second). Alternatively, temperature controllers can change temperature at a non-linear rate. Temperature controllers can increase or decrease temperature.

The reagents and reporter molecule constructs may enable the detection of the analytes by the sensor array according to a specific assay methodology. The digital or computer system may coordinate the operation of one or more components of the system, such as collecting data, communicating the data to a processing or analysis unit, or both collecting and communicating the data.

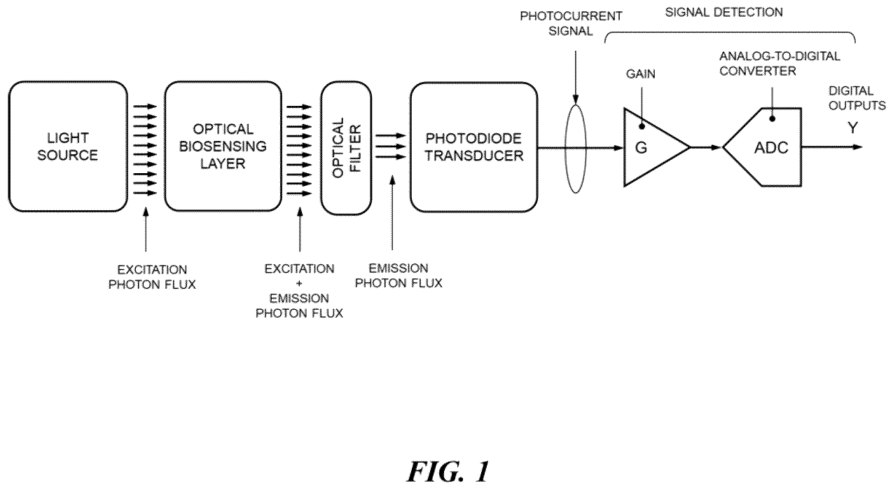

Systems and devices described herein may include a light source, optical sensing layer (e.g., surface layer), filters, transducers, detection circuitry, or any combination thereof. FIG. 1 shows an example block diagram of a sensor system using fluorescent-based transductions methods. The system may include a light source that generates an excitation photon flux and directs the photon flux to a sensing layer, for example, an optical sensing layer. The optical sensing layer may comprise an interaction moiety (e.g., capture probe) configured to bind or otherwise interact with the analyte to generate an optical signal. The optical sensing layer may generate an emission photon flux that may be filtered using one or more optical filters. The optical filters may filter excitation and/or scattered light to prevent the light from reaching the photodiode transducer. The photodiode transducer may convert the optical signal to an electrical (e.g., photocurrent) signal. The electrical current may be directed to detection circuitry (e.g., gain, analog-to-digital converters, etc.) configured to generate a digital output signal.

A sensor in an array of sensors may include a photodiode transducer. The photodiode transducer may be a differential photodiode transducer. A differential photodiode transducer may be an optical transducer that comprises at least two photodiode elements (e.g., photodiodes). The differential photodiode transducer may include at least 2, 3, 4, 5, 6, 8, 10, or more photodiodes. In an example, the differential photodiode transducer comprises two photodiodes. The photodiodes may be the identical or may be different. In an example, the photodiodes are identical. The first photodiode may be configured to receive and transduce incident photons to an electrical signal (e.g., electrons) from the array and the sample. The first photodiode may be a bright photodiode as it is configured to receive or as it receives incident photons from the light source and sample. A second photodiode may comprise an optical cover. The optical cover may be configured to reduce or block or may reduce or block incident photons from contacting the second photodiode. As such, the second photodiode may be a “dark” photodiode.

The cover may comprise one or more metals. The one or more metals may include Aluminum, Copper, Tungsten, or other metals that are used in the manufacturing of semiconductor devices. The optical cover may block incident photons independent of the wavelength of the photons. For example, the optical cover may comprise a cover configured to block or that blocks all or substantially all wavelengths of light. Alternatively, the optical cover may filter the incident photons to remove selected wavelengths of incident photons. The optical cover may block greater than or equal to about 50%, 60%, 70%, 80%, 90%, 95%, 97%, 98%, 99%, or more of the incident photons. In an example, the optical cover blocks greater than or equal to about 95% of the incident photons. In another example, the optical cover blocks greater than or equal to about 99% of the incident photons. The cover may be disposed between the dark photodiode and the surface layer. The cover may be the same size or shape as the dark photodiode. The cover may have a larger surface area than the dark photodiode to block or substantially block the dark photodiode from photon flux. The cover may have a surface area that is at least about 1%, 2%, 3%, 5%, 7%, 10%, 15%, 20%, 25%, or greater larger than a surface area of the dark photodiode.

The first (e.g., bright) and second (e.g., dark) photodiode may be disposed in spatial proximity of one another. The first and second photodiode may be disposed at the same or substantially the same depth away from the surface. Alternatively, the first and second photodiode may be disposed at different depth away from the surface. In an example, the first and second photodiode are disposed at the same or substantially the same depth away from the surface such that the observed photon flux of both photodiodes is the same or substantially the same for each photodiode. The first and second photodiodes may be disposed adjacent to one another. The first and second photodiodes may be separated by a distance of less than or equal to about 1 millimeter (mm), 0.8 mm, 0.6 mm, 0.4 mm, 0.2 mm, 0.1 mm, 80 micrometers (μm), 60 μm, 40 μm, 20 μm, 10 μm, 8 μm, 6 μm, 4 μm, 2 μm, 1 μm, 800 nanometers (nm), 600 nm, 400 nm, 200 nm, 100 nm, or less. The first and second photodiodes may be separated by a distance of greater than or equal to about 100 nm, 200 nm, 400 nm, 600 nm, 800 nm, 1 μm, 2 μm, 4 μm, 6 μm, 8 μm, 10 μm, 20 μm, 40 μm, 60 μm, 80 μm, 0.1 mm, 0.2 mm, 0.4 mm, 0.6 mm, 0.8 mm, 1 mm, or greater. The first and second photodiodes may be separated by a distance from about 100 nm to 200 nm, 100 nm to 400 nm, 100 nm to 600 nm, 100 nm to 800 nm, 100 nm to 1 μm, 100 nm to 2 μm, 100 nm to 4 μm, 100 nm to 6 μm, 100 nm to 8 μm, 100 nm to 10 μm, 100 nm to 20 μm, 100 nm to 40 μm, 100 nm to 60 μm, 100 nm to 80 μm, 100 nm to 0.1 mm, 100 nm to 0.2 mm, 100 nm to 0.4 mm, 100 nm to 0.4 mm, 100 nm to 0.6 mm, 100 nm to 0.8 mm, 100 nm to 1 mm, 200 nm to 400 nm, 200 nm to 600 nm, 200 nm to 800 nm, 200 nm to 1 μm, 200 nm to 2 μm, 200 nm to 4 μm, 200 nm to 6 μm, 200 nm to 8 μm, 200 nm to 10 μm, 200 nm to 20 μm, 200 nm to 40 μm, 200 nm to 60 μm, 200 nm to 80 μm, 200 nm to 0.1 mm, 200 nm to 0.2 mm, 200 nm to 0.4 mm, 200 nm to 0.4 mm, 200 nm to 0.6 mm, 200 nm to 0.8 mm, 200 nm to 1 mm, 400 nm to 600 nm, 400 nm to 800 nm, 400 nm to 1 μm, 400 nm to 2 μm, 400 nm to 4 μm, 400 nm to 6 μm, 400 nm to 8 μm, 400 nm to 10 μm, 400 nm to 20 μm, 400 nm to 40 μm, 400 nm to 60 μm, 400 nm to 80 μm, 400 nm to 0.1 mm, 400 nm to 0.2 mm, 400 nm to 0.4 mm, 400 nm to 0.4 mm, 400 nm to 0.6 mm, 400 nm to 0.8 mm, 400 nm to 1 mm, 600 nm to 800 nm, 600 nm to 1 μm, 600 nm to 2 μm, 600 nm to 4 μm, 600 nm to 6 μm, 600 nm to 8 μm, 600 nm to 10 μm, 600 nm to 20 μm, 600 nm to 40 μm, 600 nm to 60 μm, 600 nm to 80 μm, 600 nm to 0.1 mm, 600 nm to 0.2 mm, 600 nm to 0.4 mm, 600 nm to 0.4 mm, 600 nm to 0.6 mm, 600 nm to 0.8 mm, 600 nm to 1 mm, 800 nm to 1 μm, 800 nm to 2 μm, 800 nm to 4 μm, 800 nm to 6 μm, 800 nm to 8 μm, 800 nm to 10 μm, 800 nm to 20 μm, 800 nm to 40 μm, 800 nm to 60 μm, 800 nm to 80 μm, 800 nm to 0.1 mm, 800 nm to 0.2 mm, 800 nm to 0.4 mm, 800 nm to 0.4 mm, 800 nm to 0.6 mm, 800 nm to 0.8 mm, 800 nm to 1 mm, 1 μm to 2 μm, 1 μm to 4 μm, 1 μm to 6 μm, 1 μm to 8 μm, 1 μm to 10 μm, 1 μm to 20 μm, 1 μm to 40 μm, 1 μm to 60 μm, 1 μm to 80 μm, 1 μm to 0.1 mm, 1 μm to 0.2 mm, 1 μm to 0.4 mm, 1 μm to 0.4 mm, 1 μm to 0.6 mm, 1 μm to 0.8 mm, 1 μm to 1 mm, 2 μm to 4 μm, 2 μm to 6 μm, 2 μm to 8 μm, 2 μm to 10 μm, 2 μm to 20 μm, 2 μm to 40 μm, 2 μm to 60 μm, 2 μm to 80 μm, 2 μm to 0.1 mm, 2 μm to 0.2 mm, 2 μm to 0.4 mm, 2 μm to 0.4 mm, 2 μm to 0.6 mm, 2 μm to 0.8 mm, 2 μm to 1 mm, 4 μm to 6 μm, 4 μm to 8 μm, 4 μm to 10 μm, 4 μm to 20 μm, 4 μm to 40 μm, 4 μm to 60 μm, 4 μm to 80 μm, 4 μm to 0.1 mm, 4 μm to 0.2 mm, 4 μm to 0.4 mm, 4 μm to 0.4 mm, 4 μm to 0.6 mm, 4 μm to 0.8 mm, 4 μm to 1 mm, 6 μm to 8 μm, 6 μm to 10 μm, 6 μm to 20 μm, 6 μm to 40 μm, 6 μm to 60 μm, 6 μm to 80 μm, 6 μm to 0.1 mm, 6 μm to 0.2 mm, 6 μm to 0.4 mm, 6 μm to 0.4 mm, 6 μm to 0.6 mm, 6 μm to 0.8 mm, 6 μm to 1 mm, 8 μm to 10 μm, 8 μm to 20 μm, 8 μm to 40 μm, 8 μm to 60 μm, 8 μm to 80 μm, 8 μm to 0.1 mm, 8 μm to 0.2 mm, 8 μm to 0.4 mm, 8 μm to 0.4 mm, 8 μm to 0.6 mm, 8 μm to 0.8 mm, 8 μm to 1 mm, 10 μm to 20 μm, 10 μm to 40 μm, 10 μm to 60 μm, 10 μm to 80 μm, 10 μm to 0.1 mm, 10 μm to 0.2 mm, 10 μm to 0.4 mm, 10 μm to 0.4 mm, 10 μm to 0.6 mm, 10 μm to 0.8 mm, 10 μm to 1 mm, 20 μm to 40 μm, 20 μm to 60 μm, 20 μm to 80 μm, 20 μm to 0.1 mm, 20 μm to 0.2 mm, 20 μm to 0.4 mm, 20 μm to 0.4 mm, 20 μm to 0.6 mm, 20 μm to 0.8 mm, 20 μm to 1 mm, 40 μm to 60 μm, 40 μm to 80 μm, 40 μm to 0.1 mm, 40 μm to 0.2 mm, 40 μm to 0.4 mm, 40 μm to 0.4 mm, 40 μm to 0.6 mm, 40 μm to 0.8 mm, 40 μm to 1 mm, 60 μm to 80 μm, 60 μm to 0.1 mm, 60 μm to 0.2 mm, 60 μm to 0.4 mm, 60 μm to 0.4 mm, 60 μm to 0.6 mm, 60 μm to 0.8 mm, 60 μm to 1 mm, 80 μm to 0.1 mm, 80 μm to 0.2 mm, 80 μm to 0.4 mm, 80 μm to 0.4 mm, 80 μm to 0.6 mm, 80 μm to 0.8 mm, 80 μm to 1 mm, 0.1 mm to 0.2 mm, 0.1 mm to 0.4 mm, 0.1 mm to 0.4 mm, 0.1 mm to 0.6 mm, 0.1 mm to 0.8 mm, 0.1 mm to 1 mm, 0.2 mm to 0.4 mm, 0.2 mm to 0.4 mm, 0.2 mm to 0.6 mm, 0.2 mm to 0.8 mm, 0.2 mm to 1 mm, 0.4 mm to 0.6 mm, 0.4 mm to 0.8 mm, 0.4 mm to 1 mm, 0.6 mm to 0.8 mm, 0.6 mm to 1 mm, or 0.8 mm to 1 mm. In an example, the first and second photodiode are in the same plane and separated by a distance of about 100 nm to about 1 mm such that each photodiode observes the same excitation photon flux during the sensing process.

The differential photodiode transducer may be configured to generate or may generate two electrical outputs, one corresponding to the bright photodiode and one corresponding to the dark photodiode (e.g., covered photodiode). When considering the photocurrent output of the bright photodiode I_{DB} and dark photodiode I_{DD}, the output of the differential photodiode transducer, I₀, may be the difference between the photocurrent from the dark photodiode and the photocurrent from the bright photodiode, as shown in Equation (5):

$${I}_{0} = {I}_{DB} - {I}_{DD} = {F}_{X} ( {θ}_{X} + n {θ}_{Y} {θ}_{E} ) - α {F}_{X} ( {θ}_{X} + n {θ}_{Y} {θ}_{E} ) = n {F}_{X} ( {θ}_{X} + n {θ}_{Y} {θ}_{E} ) ( 1 - α ) \qquad ( 5 )$$

where a is the probability of photons passing through the optical cover of the dark photodiode. In an ideal case, where the cover blocks all photons, a→0, and Equation (5) may be written as shown in Equation (6):

$${I}_{0} \approx n {F}_{X} {θ}_{Y} {θ}_{E} + {F}_{X} {θ}_{X} \qquad ( 6 )$$

The optical cover may be ideal or non-ideal. In either case, Equation (5) and (6) may be independent of I_{dc}. This is an advantage of using a differential photodiode transducer as comparted to a single photodiode transducer configuration as is may eliminate or replace CDS methods and, thus, increase measurement speed. Further, the differential sensor circuitry, rather than single-ended circuitry, may be used to detect and quantize the photodiode output, which may further simplify the design of the photodetection in the CMOS chips and desensitize the sensor to on-chip process and voltage variations.

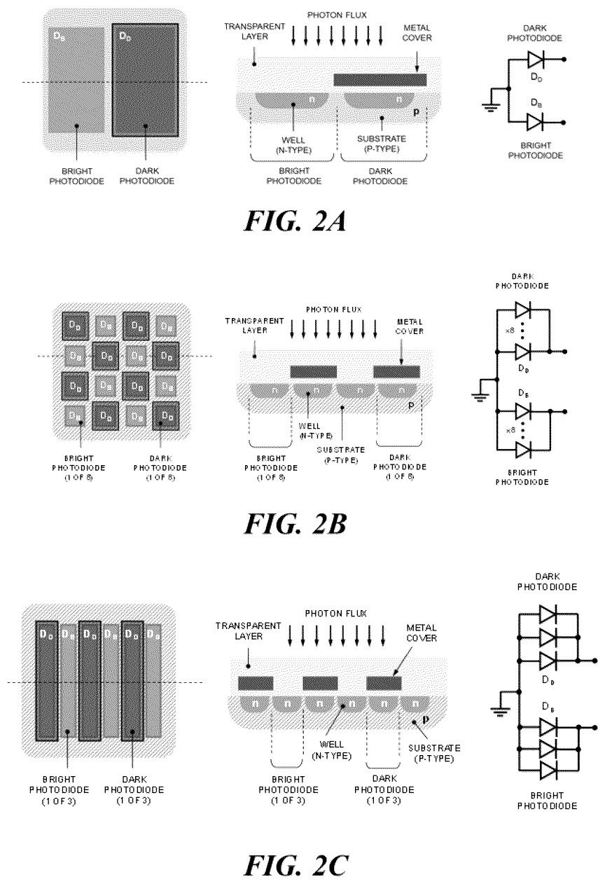

A sensing array may include a plurality of individual sensors. An individual sensor may comprise a differential photodiode comprising a first and a second photodiode as described elsewhere herein. FIGS. 2A-2C show example differential photodiode transducers and example circuit schematic. FIG. 2A shows an example differential photodiode transducer with a bright photodiode disposed adjacent to a dark photodiode. The differential photodiode transducer may be disposed below a transparent layer. The dark photodiode may have a metal cover disposed between the light source and the dark photodiode. The bright photodiode and the dark photodiode may both generate and electrical output signal. FIG. 2B shows an example sensor array comprising a grid of alternating bright and dark photodiodes. The photodiodes shown are fabricated as a 2D square disposed under a transparent layer. Each dark photodiode may include a blocking cover disposed between the dark photodiode and the light source. Each sensor (e.g., pixel) may include a bright and dark photodiode and each bright photodiode and dark photodiode may generate an electrical output signal. FIG. 2C shows an alternative example configuration of a sensor array. The photodiodes may have a 2D bar or rectangular configuration, with alternating bright and dark photodiodes each configured to generate an electrical output. The dark photodiodes may each include a cover disposed between the dark photodiode and the light source. The example sensor arrays illustrated in FIGS. 2A-2C may be fabricated using a planar semiconductor manufacturing process. The layers and materials shown in FIGS. 2A-2C may be similar to those used in CMOS fabrication processes. In FIGS. 2A-2C D_{D} and D_{B} represent the electrical outputs of the dark photodiode and bright photodiode, respectively.

Systems for fluorescent-based analyte detection may include an excitation light source. The excitation light source may be configured to deliver or may deliver a pulsed light as an excitation source. The pulsed light excitation source may permit time-resolved, also referred to as time-gated, fluorescence detection by applying a finite-time pulsed light to the sensor array. Emitted photons may be detected after the end of the pulse (e.g., in the absence of excitation light). Time-resolved detection may be compatible with a variety of different fluorophores and fluorescent reporters. See References 8 and 12. Example fluorophores may have long emission lifetimes, such as, for example metal chelates (e.g., lanthanide chelates). The pulsed light source may be a light emitted diode (LED), laser diode (LD), or non-solid state-based laser. The system may include triggering electrical circuitry to permit fast turn off of the excitation photon flux. Using time-resolved detection in combination with pulsed excitation light may permit the sensor array to detect an analyte without the use of emission filters. As such, the fluorescent-based detection system described herein may not use an emission filter for the detection of an analyte.

Fluorophores with long lifetimes may be used for time-resolved fluorescence detection. For example, the lifetime, τ_{F}, of the fluorophore may be much larger than the turn off time, t_{X}, of the pulsed photon flux. In this example, for t≥t_{X}, the output of the differential photodiode may be represented by Equation (7):

$${I}_{0} ( t ) = n {F}_{X} {θ}_{Y} {θ}_{E} exp ( - \frac{t}{{τ}_{F}} ) \qquad ( 7 )$$

which includes neither the dark current nor any background generated by the excitation photon flux. The differential photodiode output, I₀(t), may be integrated over a time window (e.g., between t₁ and t₂). The output may then be used to estimate the fluorophore concentration, n. Setting the excitation photon flux to zero at tₓ may reduce the photon emission such that photon emission may become negligible at t≥t_{X}. The reduction in photon emission may be greater than or equal to about 10⁶, 10⁷, 10⁸, 10⁹ or more. The turn off time, tₓ, may be less than or equal to about 10⁻³, 10⁻⁴, 10⁻⁵, 10⁻⁶, 10⁻⁷, 10⁻⁸, 10⁻⁹, or fewer seconds, depending on the fluorophore construct that is used.

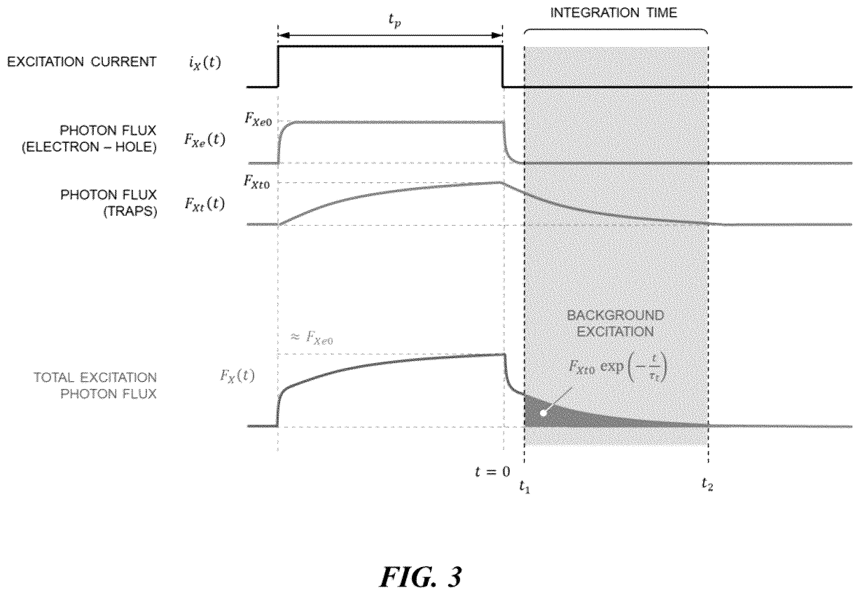

Turning off the light source completely may be challenging. For example, in an LED-based pulsed light source the photon flux, F_{X}, may include a photon flux generated from electron and hole recombination, F_{Xe}, and the photon generated by the relaxation of the deep traps within the semiconductor crystal structure, F_{Xt}, (e.g., F_{X}=F_{Xe}+F_{Xt}, where F_{Xe}>>F_{Xt}). As shown in FIG. 3, the photon flux generated from electron and hole recombination may be quickly turned off by removing the current and the excess carriers. The decay time for photons generated by the relaxation of deep traps may have a longer lifetime, τₜ, as shown in FIG. 3. For example, for LEDs, τₜ, may be greater than or equal to about 10⁻⁶, 10⁻⁵, 10⁻⁴, 10⁻³, 10⁻², 10⁻¹, or greater depending on semiconductor crystal structure. As such, background excitation photons may be present for a time period after current is turned off and Equation (7) may be written as Equation (8):

$${I}_{0} ( t ) = n {F}_{X} {θ}_{Y} {θ}_{E} exp ( - \frac{t}{{τ}_{F}} ) + ϵ {F}_{Xt 0} exp ( - \frac{t}{{τ}_{t}} ) , \qquad ( 8 )$$

where index zero may indicate the value at the end of the of the excitation pulse and ε is F_{Xt} to photocurrent conversion gain.

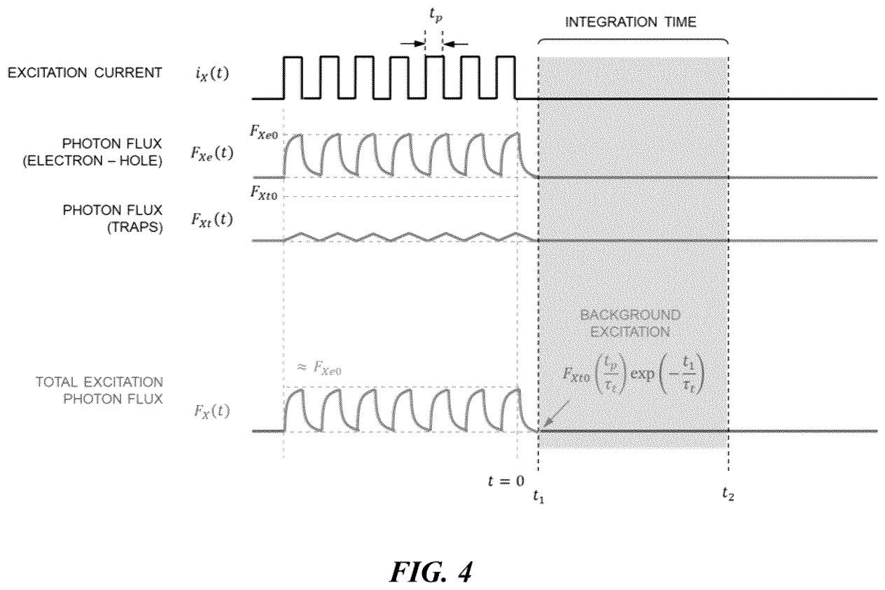

In an example, the excitation photon flux may be modulated to suppress the filling of the long lifetime traps in the semiconductor crystal structure. An example modulation scheme for a pulse-modulated light source is shown in FIG. 4. As shown in FIG. 4, the excitation photon flux may include a series of pulses, wherein a duration of each pulse is tₚ. If the duty cycle of the pulse duration, β, is less than 50% and the pulse duration is much less than the lifetime of the deep traps (e.g., tₚ<<τₜ), Equation (8) may be rewritten as Equation (9):

$${I}_{0} ( t ) = n β {F}_{X} {θ}_{Y} {θ}_{E} exp ( - \frac{t}{{τ}_{F}} ) + ϵ {F}_{Xt 0} ( \frac{{t}_{p}}{{τ}_{t}} ) exp ( - \frac{t}{{τ}_{t}} ) \approx n β {F}_{X} {θ}_{Y} {θ}_{E} exp ( - \frac{t}{{τ}_{F}} ) \qquad ( 9 )$$

The method may include directing a series of pulses from the excitation source towards the surface layer for a set period of time, halting the pulses, and taking one or more measurements from the sensing array (e.g., from a single sensor in the array or from multiple sensors across the array). The set period of time (e.g., duration of time which the excitation light source is pulsed prior to taking a measurement) may be greater than or equal to about 10 ns to 100 ms. The series of pulses may include at least about 2, 4, 6, 8, 10, 15, 20, 30, 40, 50, 60, 80, 100, 1000 or more pulses. In an example, the series of pulses includes at least 10 pulses. An individual pulse may have a duration of less than or equal to about 10⁻¹, 10⁻², 10⁻³, 10⁻⁴, 10⁻⁵, 10⁻⁶, 10⁻⁷, 10⁻⁸, 10⁻⁹ or less. The duration of time that the light source is active may be the same as the duration of time that the light source is inactive during a series of pulses. Alternatively, or in addition to, the pulse duration may be greater than or less than the non-pulse duration (e.g., when the light source is off) during a series of pulses.

The systems, devices, and methods described herein may be used with any class of fluorophore as the reporter molecule for the sensing process. The optical signal may be generated by a fluorescent reporter molecule associated with the analyte of the immobilized capture probe. A fluorophore may have a predefined and non-zero relaxation lifetime, τ_{F}. See Reference 14. With select excitation pulsing and detection timing, measurements of the decaying emissions may be taken once the photon flux is halted. Fluorophores with longer fluorescence lifetimes may enable more options for excitation pulsing and detection timing, making sensing easier. Additionally, longer relaxation lifetimes may permit background autofluorescence from biological materials (e.g., endogenous fluorophores such as melanin, collagen, etc.) to be ignored as such materials may have lifetimes of less than 10 nanoseconds (ns). The fluorophore lifetime may be greater than or equal to about 10 ns, 20 ns, 30 ns, 40 ns, 50 ns, 60 ns, 80 ns, 100 ns, 150 ns, 200 ns, 300 ns, 400 ns, 500 ns, 600 ns, 700 ns, 800 ns, 900 ns, 1 microsecond (μs), 2 μs, 3 μs, 4 μs, 5 μs, 6 μs, 7 μs, 8 μs, 9, μs, 10 μs, 20 μs, 30 μs, 40 μs, 50 μs, 60 μs, 70 μs, 80 μs, 90 μs, 100 μs, 200 μs, 300 μs, 400 μs, 500 μs, 600 μs, 700 μs, 800 μs, 900 μs, 1 millisecond (ms) or greater. The fluorophore lifetime may be about 10 ns to about 20 ns, about 20 ns to about 30 ns, about 30 ns to about 40 ns, about 40 ns to about 50 ns, about 50 ns to about 60 ns, about 60 ns to about 80 ns, about 80 ns to about 100 ns, about 100 ns to about 150 ns, about 150 ns to about 200 ns, about 200 ns to about 300 ns, about 300 ns to about 400 ns, about 400 ns to about 500 ns, about 500 ns to about 600 ns, about 600 ns to about 700 ns, about 700 ns to about 800 ns, about 800 ns to about 900 ns, about 900 ns to about 1 microsecond (μs), about 1 μs to about 2 μs, about 2 μs to about 3 μs, about 3 μs to about 4 μs, about 4 μs to about 5 μs, about 5 μs to about 6 μs, about 6 μs to about 7 μs, about 7 μs to about 8 μs, about 8 μs to about 9, μs, about 9 μs to about 10 μs, about 10 μs to about 20 μs, about 20 μs to about 30 μs, about 30 μs to about 40 μs, about 40 μs to about 50 μs, about 50 μs to about 60 μs, about 60 μs to about 70 μs, about 70 μs to about 80 μs, about 80 μs to about 90 μs, about 90 μs to about 100 μs, about 100 μs to about 200 μs, about 200 μs to about 300 μs, about 300 μs to about 400 μs, about 400 μs to about 500 μs, about 500 μs to about 600 μs, about 600 μs to about 700 μs, about 700 μs to about 800 μs, about 800 μs to about 900 μs, and about 900 μs to about 1 millisecond (ms). In an example, the fluorophore lifetime is greater than 100 ns. Non-limiting examples of fluorophores with fluorescent lifetimes of greater than 100 ns include organometallic complexes. In an example, the fluorophore lifetime is greater than 100 μs. Non-limiting examples of fluorophores with fluorescent lifetimes of greater than 100 μs include lanthanide chelates. See Reference 14. The fluorescent reporter molecules may include donor molecules and acceptor molecules. In an example, both the donor molecule and the accepter molecule may be long lifetime fluorescent molecules. In another example, the donor molecule may be a long lifetime fluorophore and the acceptor molecule may be a shorter lifetime or non-radiating acceptor fluorophore. The donor molecule and the acceptor molecule may be bound or otherwise associated with the capture probe and analyte, respectively, to permit real-time binding measurements. Alternatively, the donor molecule and the acceptor molecule may be bound or otherwise associated with the analyte and the capture probe, respectively.

The system, and corresponding methods, may be configured for detection of emission signals of a single wavelength or multiple wavelengths (e.g., multi-color capabilities). Differentiating fluorophores may be permitted by differences in fluorescence lifetimes after excitation. In some examples, fluorophores may be reactive or conjugated dyes, nucleic acid dyes, fluorescent proteins, cell function dyes, or any combination thereof. Experimental designs for the multiplex detection of multiple fluorophores are possible without the use of emission and excitation filter sets. Accordingly, multiple fluorophores can be detected in a single experiment by the differential electrical output signals of the bright and dark photodiodes in the absence of excitation and emission filter sets. Individual species of fluorophores may be detected based on the differences in their decay rates. For example, metal chelate, such as Lanthanide chelates may be used as time-resolved fluorophores. In some cases, time-resolved fluorophores may act as molecular reporters in time-resolved assays either as a standalone reporter or an element (donor or acceptor) in a fluorescence energy transfer moiety. Examples include, but are not limited to, Forster Resonance Energy Transfer (FRET) technologies. The role of time-resolved fluorophores may include facilitating the generation of a specific time-resolved fluorescent signal that may be correlated to the presence or absence of a molecular reaction or presence or absence of a specific target analyte. Time-resolved fluorophores may be used as labels for specific target analytes, in applications where the targets may be chemically modified to incorporate a time-resolved fluorophore. Examples includes, but are not limited to, Northern blots, Southern blots, DNA microarrays, quantitative Polymerase Chain Reaction (PCR), digital PCR, and diagnostic assays. In microarrays and Norther blots, the mRNA target analyte may be converted into a fluorophore-labelled complementary DNA (cDNA), for example, through reverse transcription. In Southern blots, a fluorophore-labeled cDNA may be used to identify a target sequence. In quantitative PCR and digital PCR, the fluorophore may be incorporated into an amplified nucleic acid sequence or a primer sequence to demonstrate the accumulation of a target sequence. In a diagnostic assay, a device may be used to sequester target nucleic acids, and a fluorophore-labelled cDNA may be used for direct detection.

Time-resolved fluorophores may be used as labels for the detection of probes in a sandwich assay, Non-limiting examples include Western Blots, Enzyme-Linked Immunosorbent Assay (ELISA), Enzyme-Linked Immuno SPOT (ELISPOT), FluoroSpot assay, protein arrays, or any combination thereof. In sandwich assays, the time-resolved fluorophores may be used as a direct method for detection, in which the fluorophore is conjugated to a primary detection antibody. Alternatively, or in addition to, the time-resolved fluorophore may be used as an indirect method for detection, for example, the fluorophore may be conjugated to a secondary antibody. ELISPOT assays may be used to quantitatively measure the frequency of cytokine secretion for a single cell. The ELISASPOT assay may be a form of immunostaining that uses antibodies to detect an analyte, including but not limited to, any biological or chemical substance (e.g., protein analytes or chemical analytes) FluoroSpot assays may use fluorescence to analyze multiple analytes, for example, by detecting the secretion of more than one type of protein or other analytes.

Time-resolved fluorophores may be used as labels in cell sorting, counting, or detecting methods. An example may be flow cytometry, in chick cells may be labeled with a fluorophore. For example, cells may be sorted and counted by their fluorescence profiles. Alternatively, or in addition to, the specific cellular characteristics or functions may be identified by their fluorescence profiles.

Time-resolved fluorophores may be used in application where solid-phase and immobilized capture probes are labeled. An example may include inverse fluorophore assays. Time-resolved fluorophores may be used in assays in which chemical reactions may be monitored while a target analyte is introduced a reacting reagent. The target molecule or the reacting reagent may include time-resolved fluorophores. Examples may include, but are not limited to, Sanger Sequencing and Next Generation Sequencing (NGS) assays such as sequence-by-synthesis (SBS), and pyrosequencing.

The fluorescent-based sensing device may further include a current switch. A current switch may divert the output current, I₀, from the photodiode transducer to dissimilar current detection paths. For example, the current switch may divert the output current from the photodiode transducer to at least 2, 3, 4, 5, 6, 7, 8, 9, 10, or more detection paths. In an example, the current switch may direct the output current to at least two different detection paths. The first detection path may be a high gain detection path and the second detection path may be a low gain detection path. The current switch may direct the output current to the high gain detection path when the excitation light source is off (e.g., when excitation photon flux is zero). The current switch may direct the output current to the low gain detection path when the light pulse is active (e.g., when excitation photon flux is greater than zero). In an example, the sensing device does not include an emission filter and so, when the photon flux is active, the output signal of the differential photodiode may be dominated by the excitation source, as shown in Equation (10):

$${I}_{0} ( t ) \approx {F}_{X} {θ}_{X} \qquad ( 10 )$$

which may permit I₀(t) to represent the excitation photon flux, F_{X}, and the amplitude of the excitation light at an individual sensor (e.g., pixel). This estimated value may be used in conjunction with the output of the high gain path formulated by Equations (7) and (9) to estimate the fluorophore concentration, n. This approach may not be usable with a sensing system that includes emission filters as the excitation photon flux is blocked by the emission filter, which may make the low gain measurements non-informative.

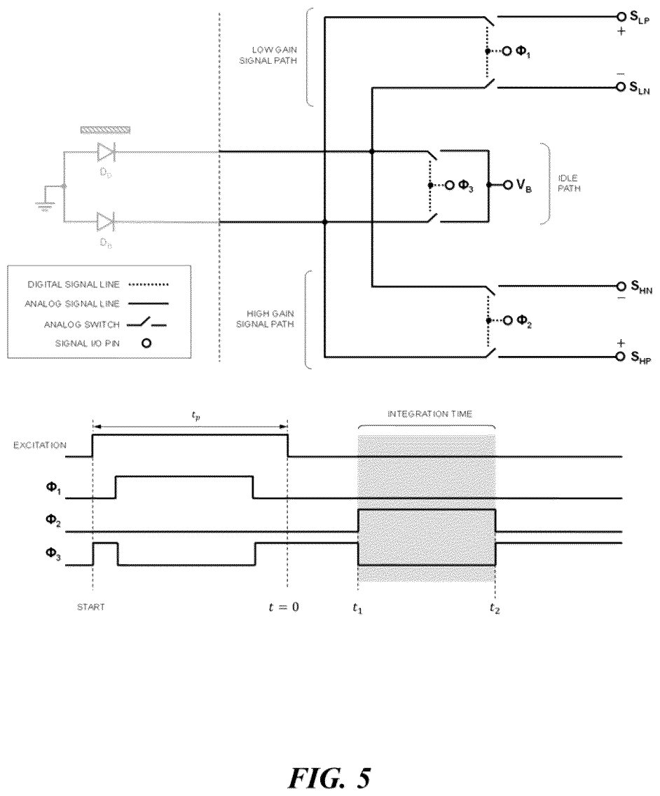

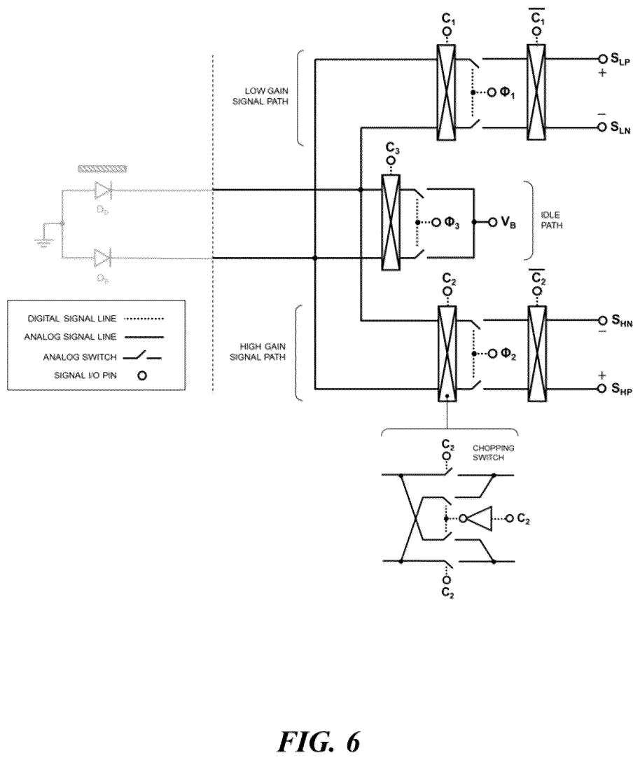

Example current switches and corresponding timing diagram are shown in FIG. 5. FIG. 5 illustrates an example system including a photodiode transducer connected to circuitry including three possible current pathways, such as a low gain signal detection path with outputs represented by S_{LP} and S_{LN}, high gain signal detection path with outputs represented by S_{HP} and S_{HN}, and idle path with may be terminated to bias voltage V_{B}. As shown in the timing diagram, the low gain path, ϕ₁, may be activated to measure the excitation photon flux and the high gain path, ϕ₂, may be activated between a timepoint t₁ and t₂ to measure the emission signal. When neither the low gain nor high gain paths are activated, the system may be connected to an idle path ϕ₃. An alternative current switch configuration is shown in FIG. 6. The example current switch includes chopper stabilization switches which may be configured to suppress or reduce offset current, unbalanced charge injection, or both. See Reference 13.

The sensor array may include a plurality of sensors (e.g., a plurality of pixels). The sensor array may include greater than or equal to about 10, 10², 10³, 10⁴, 10⁵, 10⁶, 10⁷, 10⁸, 10⁹ or more sensors (e.g., pixels). The sensor array may include from about 10 to 10², 10 to 10³, 10 to 10⁴, 10 to 10⁵, 10 to 10⁶, 10 to 10⁷, 10 to 10⁸, 10 to 10⁹, 10², to 10³, 10² to 10⁴, 10² to 10⁵, 10² to 10⁶, 10² to 10⁷, 10² to 10⁸, 10² to 10⁹, 10³ to 10⁴, 10³ to 10⁵, 10³ to 10⁶, 10³ to 10⁷, 10³ to 10⁸, 10³ to 10⁹, 10⁴ to 10⁵, 10⁴ to 10⁶, 10⁴ to 10⁷, 10⁴ to 10⁸, 10⁴ to 10⁹, 10⁵ to 10⁶, 10⁵ to 10⁷, 10⁵ to 10⁸, 10⁵ to 10⁹, 10⁶ to 10⁷, 10⁶ to 10⁸, 10⁶ to 10⁹, 10⁷ to 10⁸, 10⁷ to 10⁹, or 10⁸ to 10⁹ sensors. The sensors may be evenly spaced or the spacing of the sensors may vary across the sensor array. A sensor may be disposed adjacent to another sensor. A sensor may be separated from another sensor by a distance. The distance separating two sensors may be less than or equal to about 1 mm, 0.8 mm, 0.6 mm, 0.4 mm, 0.2 mm, 0.1 mm, 80 micrometers (μm), 60 μm, 40 μm, 20 μm, 10 μm, 8 μm, 6 μm, 4 μm, 2 μm, 1 μm, 0.5 μm, 0.2 μm, 0.1 μm, or less. The distance separating two sensors may be greater than or equal to about 0.1 μm, 0.2 μm, 0.5 μm, 1 μm, 2 μm, 4 μm, 6 μm, 8 μm, 10 μm, 20 μm, 40 μm, 60 μm, 80 μm, 0.1 mm, 0.2 mm, 0.4 mm, 0.6 mm, 0.8 mm, 1 mm, or greater. The distance separating two sensors may be from about 0.1 μm to 0.2 μm, 0.1 μm to 0.4 μm, 0.1 μm to 0.6 μm, 0.1 μm to 0.8 μm, 0.1 μm to 1 μm, 0.1 μm to 2 μm, 0.1 μm to 4 μm, 0.1 μm to 6 μm, 0.1 μm to 8 μm, 0.1 μm to 10 μm, 0.1 μm to 20 μm, 0.1 μm to 40 μm, 0.1 μm to 60 μm, 0.1 μm to 80 μm, 0.1 μm to 0.1 mm, 0.1 μm to 0.2 mm, 0.1 μm to 0.4 mm, 0.1 μm to 0.6 mm, 0.1 μm to 0.8 mm, 0.1 μm to 1 mm, 0.2 μm to 0.4 μm, 0.2 μm to 0.6 μm, 0.2 μm to 0.8 μm, 0.2 μm to 1 μm, 0.2 μm to 2 μm, 0.2 μm to 4 μm, 0.2 μm to 6 μm, 0.2 μm to 8 μm, 0.2 μm to 10 μm, 0.2 μm to 20 μm, 0.2 μm to 40 μm, 0.2 μm to 60 μm, 0.2 μm to 80 μm, 0.2 μm to 0.1 mm, 0.2 μm to 0.2 mm, 0.2 μm to 0.4 mm, 0.2 μm to 0.6 mm, 0.2 μm to 0.8 mm, 0.2 μm to 1 mm, 0.4 μm to 0.6 μm, 0.4 μm to 0.8 μm, 0.4 μm to 1 μm, 0.4 μm to 2 μm, 0.4 μm to 4 μm, 0.4 μm to 6 μm, 0.4 μm to 8 μm, 0.4 μm to 10 μm, 0.4 μm to 20 μm, 0.4 μm to 40 μm, 0.4 μm to 60 μm, 0.4 μm to 80 μm, 0.4 μm to 0.1 mm, 0.4 μm to 0.2 mm, 0.4 μm to 0.4 mm, 0.4 μm to 0.6 mm, 0.4 μm to 0.8 mm, 0.4 μm to 1 mm, 0.6 μm to 0.8 μm, 0.6 μm to 1 μm, 0.6 μm to 2 μm, 0.6 μm to 4 μm, 0.6 μm to 6 μm, 0.6 μm to 8 μm, 0.6 μm to 10 μm, 0.6 μm to 20 μm, 0.6 μm to 40 μm, 0.6 μm to 60 μm, 0.6 μm to 80 μm, 0.6 μm to 0.1 mm, 0.6 μm to 0.2 mm, 0.6 μm to 0.4 mm, 0.6 μm to 0.6 mm, 0.6 μm to 0.8 mm, 0.6 μm to 1 mm, 0.8 μm to 1 μm, 0.8 μm to 2 μm, 0.8 μm to 4 μm, 0.8 μm to 6 μm, 0.8 μm to 8 μm, 0.8 μm to 10 μm, 0.8 μm to 20 μm, 0.8 μm to 40 μm, 0.8 μm to 60 μm, 0.8 μm to 80 μm, 0.8 μm to 0.1 mm, 0.8 μm to 0.2 mm, 0.8 μm to 0.4 mm, 0.8 μm to 0.6 mm, 0.8 μm to 0.8 mm, 0.8 μm to 1 mm, 1 μm to 2 μm, 1 μm to 4 μm, 1 μm to 6 μm, 1 μm to 8 μm, 1 μm to 10 μm, 1 μm to 20 μm, 1 μm to 40 μm, 1 μm to 60 μm, 1 μm to 80 μm, 1 μm to 0.1 mm, 1 μm to 0.2 mm, 1 μm to 0.4 mm, 1 μm to 0.6 mm, 1 μm to 0.8 mm, 1 μm to 1 mm, 2 μm to 4 μm, 2 μm to 6 μm, 2 μm to 8 μm, 2 μm to 10 μm, 2 μm to 20 μm, 2 μm to 40 μm, 2 μm to 60 μm, 2 μm to 80 μm, 2 μm to 0.1 mm, 2 μm to 0.2 mm, 2 μm to 0.4 mm, 2 μm to 0.6 mm, 2 μm to 0.8 mm, 2 μm to 1 mm, 4 μm to 6 μm, 4 μm to 8 μm, 4 μm to 10 μm, 4 μm to 20 μm, 4 μm to 40 μm, 4 μm to 60 μm, 4 μm to 80 μm, 4 μm to 0.1 mm, 4 μm to 0.2 mm, 4 μm to 0.4 mm, 4 μm to 0.6 mm, 4 μm to 0.8 mm, 4 μm to 1 mm, 6 μm to 8 μm, 6 μm to 10 μm, 6 μm to 20 μm, 6 μm to 40 μm, 6 μm to 60 μm, 6 μm to 80 μm, 6 μm to 0.1 mm, 6 μm to 0.2 mm, 6 μm to 0.4 mm, 6 μm to 0.6 mm, 6 μm to 0.8 mm, 6 μm to 1 mm, 8 μm to 10 μm, 8 μm to 20 μm, 8 μm to 40 μm, 8 μm to 60 μm, 8 μm to 80 μm, 8 μm to 0.1 mm, 8 μm to 0.2 mm, 8 μm to 0.4 mm, 8 μm to 0.6 mm, 8 μm to 0.8 mm, 8 μm to 1 mm, 10 μm to 20 μm, 10 μm to 40 μm, 10 μm to 60 μm, 10 μm to 80 μm, 10 μm to 0.1 mm, 10 μm to 0.2 mm, 10 μm to 0.4 mm, 10 μm to 0.6 mm, 10 μm to 0.8 mm, 10 μm to 1 mm, 20 μm to 40 μm, 20 μm to 60 μm, 20 μm to 80 μm, 20 μm to 0.1 mm, 20 μm to 0.2 mm, 20 μm to 0.4 mm, 20 μm to 0.6 mm, 20 μm to 0.8 mm, 20 μm to 1 mm, 40 μm to 60 μm, 40 μm to 80 μm, 40 μm to 0.1 mm, 40 μm to 0.2 mm, 40 μm to 0.4 mm, 40 μm to 0.6 mm, 40 μm to 0.8 mm, 40 μm to 1 mm, 60 μm to 80 μm, 60 μm to 0.1 mm, 60 μm to 0.2 mm, 60 μm to 0.4 mm, 60 μm to 0.6 mm, 60 μm to 0.8 mm, 60 μm to 1 mm, 80 μm to 0.1 mm, 80 μm to 0.2 mm, 80 μm to 0.4 mm, 80 μm to 0.6 mm, 80 μm to 0.8 mm, 80 μm to 1 mm, 0.1 mm to 0.2 mm, 0.1 mm to 0.4 mm, 0.1 mm to 0.6 mm, 0.1 mm to 0.8 mm, 0.1 mm to 1 mm, 0.2 mm to 0.4 mm, 0.2 mm to 0.6 mm, 0.2 mm to 0.8 mm, 0.2 mm to 1 mm, 0.4 mm to 0.6 mm, 0.4 mm to 0.8 mm, 0.4 mm to 1 mm, 0.6 mm to 0.8 mm, 0.6 mm to 1 mm, or 0.8 mm to 1 mm. In an example, a distance between any two sensors is from about 0.1 μm to 1 mm. In another example, a distance between any two sensors is from about 20 μm and 200 μm.

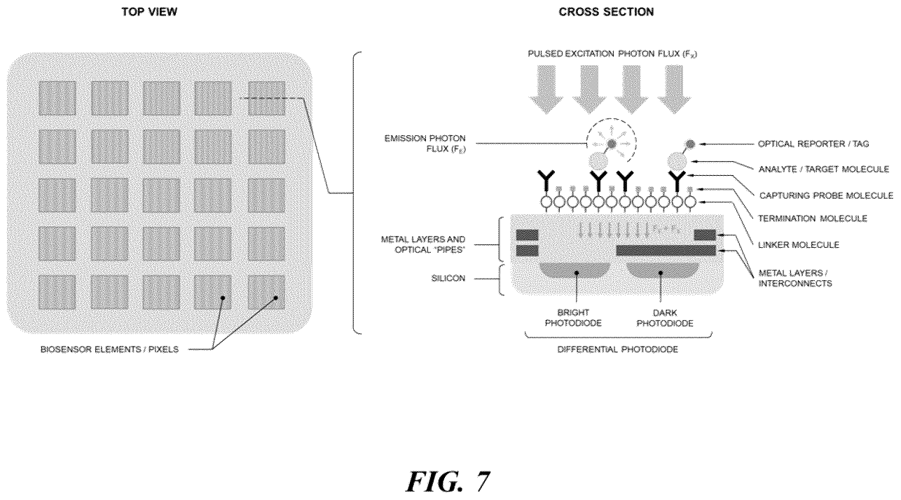

FIG. 7 shows an example top view and cross-sectional view of an example differential fluorescence-based sensor system. The system may include a 2D array of sensors. A sensor of the sensor array may include a surface layer comprising a capture probe immobilized thereon. Each sensor may be associated with or in sensing communication with a single type of capture probe configured to selectively bind a single analyte. Alternatively, a sensor may be associated with or in sensing communication with multiple types of capture probes, for example, at least 1, 2, 3, 4, 5, 6, 8, 10, or more types of capture probes configured to selectively bind at least 1, 2, 3, 4, 5, 6, 8, 10, or more different analytes. Each sensor in the sensor array may be associated with or in sensing communication (e.g., optical communication with the photodiode transducer of the sensor) with a different type of capture probe or multiple sensors may be associated with or in sensing communication with the same type of capture probe (e.g., to permit duplicate readings across the sensor array). In an example, each sensor is associated with or in sensing communication (e.g., optical communication with the photodiode transducer of the sensor) with a single type of capture probe such that each sensor detects a different analyte. In another example, at least 2, 3, 4, 5, or more sensors are associated with the same capture probe such that the sensor array generates at least 2, 3, 4, 5, or more duplicate readings across the sensor array for an analyte.

The sensing array may include a sensing layer (e.g., surface layer). The surface layer may be integrated with the sensing array and disposed on surface of the sensing array such that the surface layer is configured to contact or contact an aqueous solution comprising or suspected of comprising the analyte. The sensing layer may include an organic layer that may be created on top of the sensing array and configured to interface with a reaction chamber. The sensing layer may provide addressable locations comprising capture probes disposed on top of individual sensors and permit, by transduction of an optical signal to an electrical signal, capturing and detection of an analyte. The sensing layer may be generated by various methods. For example, capture probes or probe structures may be physically printed, immobilized, spotted, or chemically synthesized on the surface layer. In some examples, the capture probes may be randomly distributed within the 2D array surface and then identified prior to detecting the targets by alternative approaches. For example, the surface layer (e.g., silicon oxide or silicon nitride surface) of the sensor array may be chemically modified with linkers or thin film structures to permit capture probe attachment.

The capture probes may be immobilized on a surface layer of the sensor array. The sensing array may include a single type of capture probe or a plurality of different types of capture probes. The capture probes may be configured or selected to bind or interact with a targeted analyte. The capture probes may be immobilized and spatially arranged such that they are disposed in optical communication of an individual sensor. The capture probes may be disposed at independently or individually addressable locations on a solid surface of the sensing array. The capture probes may be immobilized using a covalent or non-covalent linker. In an example, the capture probes are immobilized using a covalent linker. In another example, the capture probes are immobilized using a non-covalent linker. In another example, the capture probes are immobilized using both covalent and non-covalent linkers. The linkers may be provided on the surface layer in excess of the number of capture probes. The surface layer may include 10%, 20%, 30%, 40%, 50%, or more linkers than capture probes. Unoccupied linkers (e.g., those not coupled to a capture probe) may be chemical termination to prevent or reduce interaction or interference during sensing of an analyte.

As shown in FIG. 7, a differential photodiode transducer and cover for dark photodiode may be embedded in a silicon substrate of a CMOS device. The sensor array may further comprise isolators disposed between individual sensors. The isolators may be embedded in the silicon substrate and may be configured to optically isolate a sensor from another sensor. Alternatively, or in addition to, the isolators may be configured as optical pipes that direct optical signal from the surface layer to the photodiode transducer. The system may further include a reaction chamber disposed adjacent to the surface layer of the sensor array. The reaction chamber may be configured to hold a solution comprising said analyte such that said analyte may be in contact with said capture probes immobilized on the surface layer.

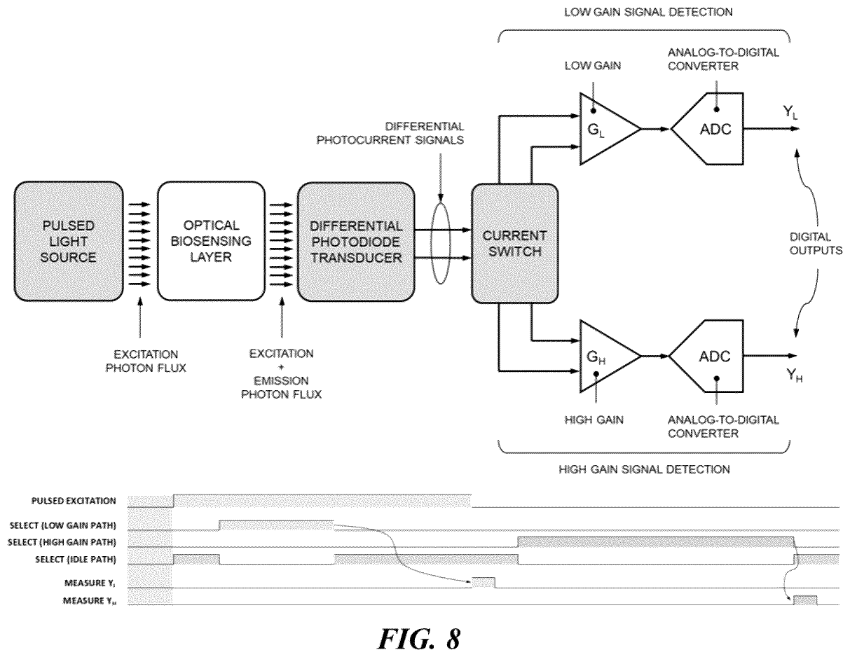

FIG. 8 shows a block diagram of an example detection circuit integrated with each sensor (e.g., pixel), and example timing diagram for the system. The system may include a photodiode transducer, such as a differential photodiode transducer, a surface layer configured to permit an analyte to interact with a capture probe, an excitation source configured to generate a pulsed excitation beam, and a current switch. The current switch may have three different detection paths, including an idle detection path (not shown). The differential photodiode transducer may produce two digital outputs, Y_{L} and Y_{H}, corresponding to the output of the low gain and high gain outputs, respectively. The system shown in FIG. 8 may be used to estimate fluorophore concentration, n, at the sensor level using Equation (10) and the electrical outputs. The low gain output may be represented by Equation (11):

$${Y}_{L} = {G}_{L} {F}_{X} {θ}_{X} \qquad ( 11 )$$

in which G_{L}=Y_{L}/I₀(t) is the gain of the low gain path. The high gain output may be determined by integrating the time decaying output, I₀(t), form Equation (9) within the time interval of [t₁, t₂] and applying the high gain of G_{H}, as shown in Equation (12):

$${Y}_{H} = {G}_{H} n {F}_{X} {θ}_{Y} {θ}_{E} {\int }_{{t}_{1}}^{{t}_{2}} exp ( - \frac{α}{{τ}_{F}} ) d α = {G}_{H} n {F}_{X} {θ}_{Y} {θ}_{E} {τ}_{F} ( exp ( - \frac{{τ}_{1}}{{τ}_{F}} ) - exp ( - \frac{{τ}_{2}}{{τ}_{F}} ) ) \qquad ( 12 )$$

the calibrated output, Y_{C}, may then be calculated by dividing the high gain output, Y_{H}, by the low gain detection path output, Y_{L}, as shown in Equation (13):

$${Y}_{C} = \frac{{Y}_{H}}{{Y}_{L}} = n ( \frac{{G}_{H}}{{G}_{L}} ) ( \frac{{θ}_{E}}{{θ}_{X}} ) ( {τ}_{F} {θ}_{Y} ) ( exp ( - \frac{{τ}_{1}}{{τ}_{F}} ) - exp ( - \frac{{τ}_{2}}{{τ}_{F}} ) ) \qquad ( 13 )$$

As shown in Equation (13), the calibrated output may not be a function of excitation photon flux, but may be a function of the fluorophore concentration, optical path constants BE/OX, fluorophore-based parameter τ_{F}θ_{Y}, detection gains G_{H}/G_{L}, and integration time.

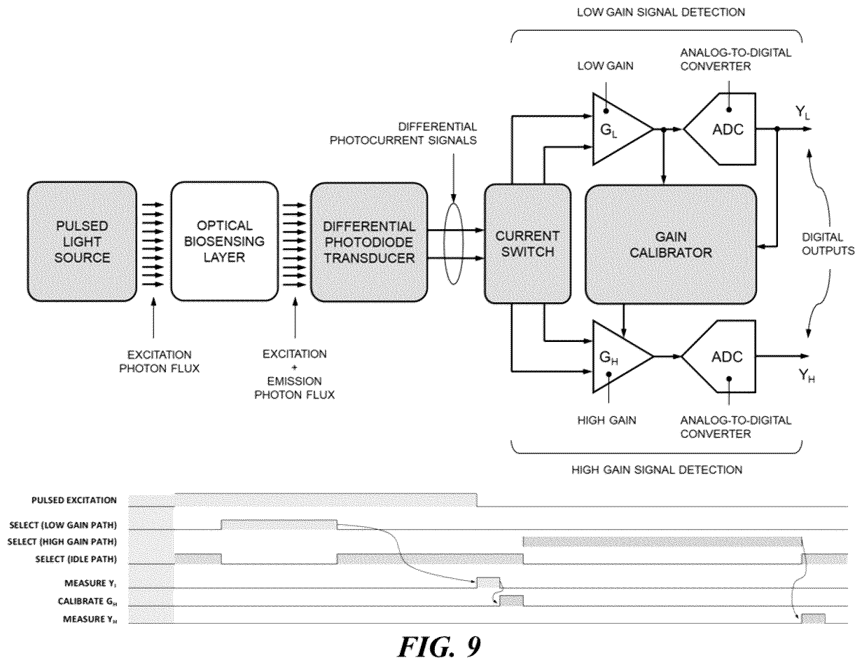

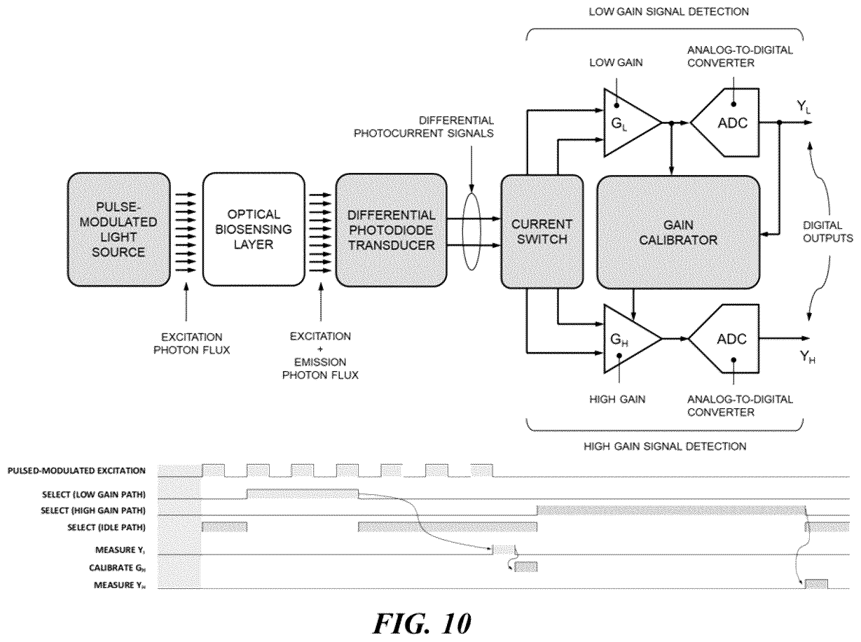

FIG. 9 shows a block diagram of an example detection circuit integrated within the sensor, and timing diagram for the system. The illustrated system includes a differential photodiode transducer, a surface layer configured for sensing, a light source configured to direct pulsed excitation light to the surface layer, a current switch with at least two separate detection paths, and a calibration block. The calibration block may be configured to adjust the gain of the high gain path according to the excitation photon flux estimated in the low gain detection path. As shown in FIG. 10, the example detection circuit may be used with a pulse-modulated excitation source.

References (each of which is incorporated by reference in its entirety)
1. Manickam, A., Singh, R., McDermott, M. W., Wood, N., Bolouki, S., Naraghi-Arani, P., Johnson, K. A., Kuimelis, R. G., Schoolnik, G. and Hassibi, A., 2017. A fully integrated CMOS fluorescence biochip for DNA and RNA testing. IEEE journal of solid-state circuits, 52(11), pp. 2857-2870.
2. Manickam, A., You, K. D., Wood, N., Pei, L., Liu, Y., Singh, R., Gamini, N., McDermott, M. W., Shahrjerdi, D., Kuimelis, R. G. and Hassibi, A., 2019. A CMOS Electrochemical Biochip with 32×32 Three-Electrode Voltammetry Pixels. IEEE Journal of Solid-State Circuits, 54(11), pp. 2980-2990.
3. Hassibi, A. et. al, 2018. Multiplexed identification, quantification and genotyping of infectious agents using a semiconductor biochip. Nature biotechnology, 36(8), pp. 738-745.
4. Hassibi, A., Hassibi, B., Vikalo, H. and Riechmann, J. L., California Institute of Technology CalTech, 2015. Real time microarrays. U.S. Pat. No. 9,133,504.
5. Hassibi, A., Vikalo, H., Riechmann, J. L. and Hassibi, B., 2009. Real-time DNA microarray analysis. Nucleic acids research, 37(20), pp. e132-e132.
6. Hassibi, A., Hassibi, B. and Vikalo, H., California Institute of Technology CalTech, 2011. Multiplex Q-PCR arrays. U.S. Pat. No. 8,048,626.
7. Hassibi, A., Jirage, K., Manickam, A. and Milaninia, K., InSilixa Inc, 2017. Multiplexed analysis of nucleic acid hybridization thermodynamics using integrated arrays. U.S. Pat. No. 9,708,647.
8. Hassibi, A., Manickam, A., Singh, R. and Kuimelis, R. G., Insilixa Inc, 2022. Methods and systems for time-gated fluorescent-based detection. U.S. Pat. No. 11,360,029.
9. Macleod, H. A. and Macleod, H. A., 2010. Thin-film optical filters. CRC press.
10. Yang, C., Shen, W., Zhang, Y., Li, K., Fang, X., Zhang, X. and Liu, X., 2015. Compact multilayer film structure for angle insensitive color filtering. Scientific reports, 5(1), pp. 1-5.
11. Yamazaki, M., Hofmann, O., Ryu, G., Xiaoe, L., Lee, T. K., deMello, A. J. and deMello, J. C., 2011. Non-emissive colour filters for fluorescence detection. Lab on a Chip, 11(7), pp. 1228-1233.
12. Shepard, K. L., Levicky, R. and Patounakis, G., Columbia University of New York, 2010. Active CMOS biosensor chip for fluorescent-based detection. U.S. Pat. No. 7,738,086.
13. Wu, R., Huijsing, J. H. and Makinwa, K. A., 2013. Dynamic offset cancellation techniques for operational amplifiers. Precision Instrumentation Amplifiers and Read-Out Integrated Circuits, pp. 21-49.
14. Berezin, M. Y. and Achilefu, S., 2010. Fluorescence lifetime measurements and biological imaging. Chemical reviews, 110(5), pp. 2641-2684.

### Computer Systems

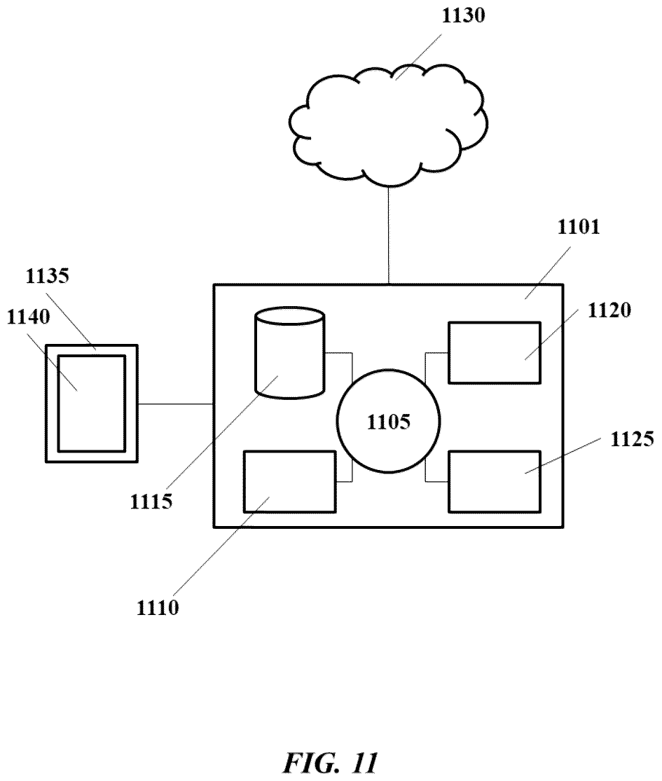

The present disclosure provides computer systems that are programmed to implement methods of the disclosure. FIG. 11 shows a computer system 1101 that is programmed or otherwise configured to implement the methods described herein, e.g., methods for detecting a presence or absence of an analyte. The computer system 1101 can regulate various aspects of excitation light generation, acquisition of output signal, and signal processing of the present disclosure, such as, for example, generating pulsed excitation light for the detection of a presence or absence of an analyte. The computer system 1101 part of a system configured for detection of an analyte. The computer system may be integrated with the detection system. Alternatively, or in addition to, the computer system may be an external computer system coupled to the detection system via wired connection or wireless connection (e.g., WiFi or Bluetooth connection).

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., laboratory technician, researcher, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, operating parameters of the system, system status, or outputs of methods described elsewhere herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, process output signals for determination of a presence or absence of an analyte.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-35. (canceled)

36. A device for time-gated detection of a presence or absence of an analyte in a solution, comprising:
a biochip comprising:
a surface layer comprising at least one immobilized capture probe specific for said analyte;
a first optical transducer in optical communication with said surface layer;
a second optical transducer disposed adjacent to said first optical transducer; and
circuitry configured to:
collect, by said first optical transducer, a first optical signal from said surface layer generated upon exposure of said surface layer to a light source, and convert said first optical signal to a first electrical signal,
collect, by said second optical transducer, a second optical signal, and convert said second optical signal to a second electrical signal, and
generate an output signal derived at least in part from a differential of said first and second electrical signals, wherein said output signal is associated with said presence or absence of said analyte.

37. The device of claim 36, wherein said light source is configured to synchronize with said biochip and emit a pulse of excitation energy, wherein:
said pulse of excitation energy comprises a first duration of time (tₚ), a duty of cycle of said pulse of excitation energy is no more than 50%;
said first optical signal comprises a fluorescence signal having a relaxation lifetime (τ_{F}); and
said first duration of time (tₚ) is about 0.1% to about 50% of said relaxation lifetime (τ_{F}).

38. The device of claim 36, wherein said light source is configured to synchronize with said biochip and emit a plurality of pulses of excitation energy, wherein:
each pulse of excitation energy of said plurality of said pulses of excitation energy comprises a first duration of time (tₚ),
a duty of cycle of said plurality of said pulses of excitation energy is no more than 50%;
said first optical signal comprises a fluorescence signal having a relaxation lifetime (τ_{F}); and
said first duration of time (tₚ) is about 0.1% to about 50% of said relaxation lifetime (τ_{F}).

39. The device of claim 36, wherein said biochip further comprising a current switch operably connected to said first optical transducer and said second optical transducer, wherein said current switch is configured to:
divert said first and second electrical signals to a low gain detection path during a first time period when said light source is on; and
divert said first and second electrical signals to a high gain detection path during a second time period when said light source is off.

40. The device of claim 36, wherein said first optical transducer and said second optical transducer are separated by a distance about 100 nanometers (nm) to about 1 millimeter (mm).

41. The device of claim 36, wherein said first optical transducer and said second optical transducer are substantially identical.

42. The device of claim 36, wherein said first optical transducer is a first photodiode, a first photogate, or a first photo-resistive device, and wherein said second optical transducer is a second photodiode, a second photogate, or a second photo-resistive device.

43. The device of claim 36, wherein said first optical transducer is a first photodiode, and wherein said second optical transducer is a second photodiode.

44. The device of claim 36, further comprising an optical cover disposed over said second optical transducer, wherein said optical cover is configured to reduce an amount of photons emitted by said light source from contacting said second optical transducer as compared to an optical transducer without said optical cover.

45. The device of claim 44, wherein said optical cover comprises a metal, wherein said metal is aluminum, copper, gold, lead, platinum, silver, tin, titanium, tungsten, or another metal, or a metal alloy that is used in the manufacturing of semiconductor devices.

46. The device of claim 44, wherein said optical cover comprises a metal alloy, wherein said metal alloy is titanium-tungsten or alloy 42.

47. The device of claim 36, wherein said surface layer comprises a linker molecule configured to immobilize said capture probe.

48. The device of claim 36, further comprising one or more optical isolators disposed adjacent to said first and/or second optical transducers, wherein said one or more optical isolators are configured to direct photons to said first and/or second optical transducers.

49. The device of claim 48, wherein said one or more optical isolators comprise a metal.

50. The device of claim 36, wherein said device does not include an emission filter and/or an optical filter.

51. The device of claim 36, wherein said biochip further comprises a differential sensor circuitry configured to detect and quantize said first and second optical signals.

52. A method for time-gated detection of a presence or absence of an analyte in a solution, comprising:
(a) directing said solution to a device comprising:
a biochip synchronized with a light source operably coupled to said biochip, said biochip comprising:
a surface layer comprising at least one immobilized capture probe specific for said analyte,
a first optical transducer in optical communication with said surface layer, and a second optical transducer disposed adjacent to said first optical transducer;
(b) collecting, by said first optical transducer, a first optical signal from said surface layer generated upon exposure of said surface layer to said light source, and converting said first optical signal to a first electrical signal;
(c) collecting, by said second optical transducer, a second optical signal, and converting said second optical signal to a second electrical signal; and
(d) generating an output signal derived at least in part from a differential of said first and second electrical signals, wherein said output signal is associated with said presence or absence of said analyte.

53. The method of claim 52, further comprising: modulating said light source and emitting a plurality of pulses of excitation energy, wherein:
each pulse of excitation energy of said plurality of said pulses of excitation energy comprises a first duration of time (tₚ),
a duty of cycle of said plurality of said pulses of excitation energy is no more than 50%;
said first optical signal comprises a fluorescence signal having a relaxation lifetime (τ_{F}); and
said first duration of time (tₚ) is about 0.1% to about 50% of said relaxation lifetime (τ_{F}).

54. The method of claim 52, further comprising:
diverting, via a current switch operably connected to said first and second optical transducers, said first and second electrical signals to a low gain detection path during a first time period when said light source is on; and
diverting, via said current switch, said first and second electrical signals to a high gain detection path during a second time period when said light source is off.

55. The method of claim 54, further comprising:
providing a low gain digital output (Y_{L}) of a first output electrical signal based in part of said low gain detection path, and
providing a high gain digital output (Y_{H}) of a second output electrical signal based in part of said high gain detection path.

56. The method of claim 55, further comprising:
providing a calibrated digital output (Y_{C}) as said output signal, wherein
$${Y}_{C} = \frac{{Y}_{H}}{{Y}_{L}}$$
wherein said calibrated digital output (Y_{C}) is substantially not a function of excitation photon flux (F_{X}) of an excitation light emitted by said light source.

57. The method of claim 52, further comprising: repeating (b)-(d) one or more times.

58. The method of claim 52, wherein said first optical signal is generated by a fluorescent reporter molecule associated with said analyte or said immobilized capture probe, wherein said fluorescent reporter molecule has a fluorescence lifetime of greater than or equal to 100 nanoseconds (ns).

59. The method of claim 52, wherein said output signal in (d) is substantially not correlated to a dark current of said first optical transducer.

60. The method of claim 52, further comprising: detecting and quantizing said first and second optical signals using a differential sensor circuitry of said biochip.

61. The method of claim 52, wherein said biochip comprises an optical cover disposed over said second optical transducer, and wherein said method further comprises reducing an amount of photons emitted by said light source from contacting said second optical transducer.
